SEQUENCE LISTING (28 sequences)

SEQ ID NO: 1 - DNA, 20 bp, Artificial Sequence
  Description of Artificial Sequence Oligonucleotide primer
ccaggacagaggccctcaac

SEQ ID NO: 2 - DNA, 92 bp, Artificial Sequence
  Description of Artificial Sequence Synthetic nucleotide primer
attgcagctagcggtggacctaagggtggcggcggttctttagttccaagaggttctggtggcggtggctctggagactccgaaaggcattt

SEQ ID NO: 3 - DNA, 48 bp, Artificial Sequence
  Description of Artificial Sequence Synthetic nucleotide primer
tccgccacctccagaacctcctccgcccctccactccacagtgatggg

SEQ ID NO: 4 - DNA, 69 bp, Artificial Sequence
  Description of Artificial Sequence Synthetic nucleotide primer
atttgcagatctttatcacaagtcttcttcagaaataagcttttgttcccagtgtttcagaaccggctc

SEQ ID NO: 5 - DNA, 99 bp, Artificial Sequence
  Description of Artificial Sequence Synthetic nucleotide primer
attgcagctagcaaaccatgtaattgtccaaaaggtgatgttaattatgcttttttgcatgctactgatcttaagggtggcggcggttctttagttcca

SEQ ID NO: 6 - DNA, 45 bp, Artificial Sequence
  Description of Artificial Sequence Synthetic nucleotide primer
ctagcggtaaaaaggttgctgcaccagcttgggctcgtatgggtc

SEQ ID NO: 7 - DNA, 51 bp, Artificial Sequence
  Description of Artificial Sequence Synthetic nucleotide primer
ctagctctcatttggttgaagctttgtatttggtttgtggtgaaagaggtc

SEQ ID NO: 8 - DNA, 45 bp, Artificial Sequence
  Description of Artificial Sequence Synthetic nucleotide primer
ttaagacccatacgagcccaagctggtgcagcaacctttttaccg

SEQ ID NO: 9 - DNA, 51 bp, Artificial Sequence
  Description of Artificial Sequence Synthetic nucleotide primer
ttaagacctctttcaccacaaaccaaatacaaagcttcaaccaaatgagag

SEQ ID NO: 10 - DNA, 24 bp, Artificial Sequence
  Description of Artificial Sequence Synthetic nucleotide primer
ggcagccccataaacacacagtat

SEQ ID NO: 11 - DNA, 20 bp, Artificial Sequence
  Description of Artificial Sequence Synthetic nucleotide primer
taatacgactcactataggg

SEQ ID NO: 12 - DNA, 52 bp, Artificial Sequence
  Description of Artificial Sequence Synthetic nucleotide primer
taccgcgcggtgaccgagctcgggcggnnsnnsgccgagtactacaataagc

SEQ ID NO: 13 - DNA, 40 bp, Artificial Sequence
  Description of Artificial Sequence Synthetic nucleotide primer
ccgcccgagctcggtcaccgcgcggtactcgcccacgtcg

SEQ ID NO: 14 - DNA, 24 bp, Artificial Sequence
  Description of Artificial Sequence Synthetic nucleotide primer
ggcagccccataaacacacagtat

SEQ ID NO: 15 - DNA, 20 bp, Artificial Sequence
  Description of Artificial Sequence Oligonucleotide primer
taatacgactcactataggg

SEQ ID NO: 16 - DNA, 1243 bp, Artificial Sequence
  Description of Artificial Sequence Synthetic nucleotide
gctagcggtggacttaagggtggcggcggttctttagttccaagaggttctggtggcggtggctctggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggcggcttgaacagcccaatgtcgccatctccctgtccaggacagaggccctcaaccaccacaacactctggtctgttcggtgacagatttctacccagccaagatcaaagtgcgctggttcaggaatggccaggaggagaagtgggggtctcatccacacagcttattaggaatggggactggaccttccaggtcctggtcatgctggagatgacccctcatcagggagaggtctacacctgccatgtggagcatcccagcctgaagagccccatcactgtggagtggaggggcggaggaggttctggaggtggcggagaagacgacattgaggccgaccacgtaggcttctatgtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctaccaatgaggctcctcaagcgactgtgttccccaagtcccctgtgctgctgggtcagcccaacacccttatctgctttgtggacaacatcttcccacctgtgatcaacatcacatggctcagaaatagcaagtcagtcacagacggcgtttatgagaccagcttcctcgtcaaccgtgaccattccttccacaagctgtcttatctcaccttcatcccttctgatgatgacatttatgactgcaaggtggagcactggggcctggaggagccggttctgaaacactgggaacaaaagcttatttctgaagaagacttgtgataaagatct

SEQ ID NO: 17 - protein, 411 aa, Artificial Sequence
  Description of Artificial Sequence Synthetic peptide
ASGGLKGGGGSLVPRGSGGGGSGDSERHFVHQFKGECYFTNGTQRIRLVTRYIYNREEYLRFDSDVGEYRAVTELGRHSAEYYNKQYLERTRAELDTACRHNYEETEVPTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRGGGGSGGGGEDDIEADHVGFYGTTVYQSPGDIGQYTHEFDGDELFYVDLDKKKTVWRLPEFGQLILFEPQGGLQNIAAEKHNLGILTKRSNFTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEQKLISEEDL

SEQ ID NO: 18 - DNA, 39 bp, Artificial Sequence
  Description of Artificial Sequence Synthetic nucleotide
ggtaaaaaggttgctgcaccagcttgggctcgtatgggt

SEQ ID NO: 19 - protein, 13 aa, Artificial Sequence
  Description of Artificial Sequence Synthetic peptide
GKKVAAPAWARMG

SEQ ID NO: 20 - DNA, 57 bp, Artificial Sequence
  Description of Artificial Sequence Synthetic nucleotide
aaaccatgtaattgtccaaaaggtgatgttaattatgcttttttgcatgctactgat

SEQ ID NO: 21 - protein, 13 aa, Artificial Sequence
  Description of Artificial Sequence Synthetic peptide
GKKVAAPVWIRMG

SEQ ID NO: 22 - protein, 19 aa, Artificial Sequence
  Description of Artificial Sequence Synthetic peptide
KPCNCPKGDVNYAFLHATD

SEQ ID NO: 23 - DNA, 45 bp, Artificial Sequence
  Description of Artificial Sequence Synthetic nucleotide
tctcatttggttgaagctttgtatttggtttgtggtgaaagaggt

SEQ ID NO: 24 - protein, 15 aa, Artificial Sequence
  Description of Artificial Sequence Synthetic peptide
SHLVEALYLVCGERG

SEQ ID NO: 25 - DNA, 54 bp, Artificial Sequence
  Description of Artificial Sequence Synthetic nucleotide primer
tacgtggtcggcctcaatgtcgtcttcaagccgccgcagggaggtggggacctc

SEQ ID NO: 26 - DNA, 76 bp, Artificial Sequence
  Description of Artificial Sequence Synthetic nucleotide primer
caatagagatctttatcacaagtcttcttcagaaataagcttttgttcattggtagctggggtgaaatttgacctc

SEQ ID NO: 27 - DNA, 51 bp, Artificial Sequence
  Description of Artificial Sequence Synthetic nucleotide primer
gaggtccccacctccctgcggcggcttgaagacattgaggccgaccacgta

SEQ ID NO: 28 - DNA, 45 bp, Artificial Sequence
  Description of Artificial Sequence Synthetic nucleotide primer
ggcggaggaggttctggaggtggcggagaagacgacattgaggcc

(19) United States
(12) Patent Application Publication
(10) Pub. No.: US 2002/0165149 A1
(43) Pub. Date: Nov. 7, 2002

(54) Mutated class II major histocompatibility proteins

(76) Inventors: David M. Kranz, Champaign, IL (US); Scott Starwalt, Urbana, IL (US); Jeffrey A. Bluestone, San Francisco, CA (US)

Correspondence Address: GREENLEE WINNER AND SULLIVAN P C, 5370 MANHATTAN CIRCLE, SUITE 201, BOULDER, CO, 80303, US

(21) Appl. No.: 10/015,536

(22) Filed: Dec. 10, 2001

Related U.S. Application Data
(60) Provisional application No. 60/254,248, filed on Dec. 8, 2000.

(51) Int. Cl. A61K 38/17; G01N 33/53; C12N 1/18; C12P 21/02; C07K 14/74

(52) U.S. Cl. 514/12; 435/7.1; 435/254.2; 530/350; 435/69.1; 435/320.1

(57) ABSTRACT

Provided are methods for directed mutagenesis and selection of MHC Class II proteins and single chain proteins with improved conformational stability and/or binding affinity for at least one cognate ligand. The improved proteins are useful for identification of T cells that are specific for the MHC Class II proteins and for treatment of disorders including autoimmune diseases, with the disorder determining the choice of the particular improved protein, peptide-protein complex or other ligand protein complex.

### CROSS REFERENCE TO RELATED APPLICATIONS

This application is a Continuation-in-Part of U.S. Provisional Application No. 60/254,248, filed Dec. 8, 2000.

### BACKGROUND OF THE INVENTION

The field of the present invention is molecular biology, in particular, as it is related to combinatorial libraries of immune cell proteins displayed on the cell surface of a recombinant host cell. More specifically, the present invention relates to a library of major histocompatibility locus proteins displayed on the surfaces of recombinant yeast cells, to mutant MHC Class II and proteins selected for improved binding to particular target peptides, to mutant MHC proteins selected for binding to a particular antigen, to MHC Class II proteins of improved stability and to the use of the selected high affinity and/or more stable MHC derivatives in diagnostic methods and imaging assays, among other applications including prophylactic and therapeutic treatments.

Proteins encoded by the major histocompatibility complex (called MFHC proteins) are requisite components of the antigenic complexes that are involved in many diseases. These diseases include cases where the body reacts with one's own MHC proteins (in various autoimmune diseases) or infectious diseases and cancer, where the MHC are critical in binding and presenting foreign, antigenic peptides. In this invention, MHC proteins the class HI type were expressed as heterologous, surface-linked fusions on yeast cells with the goal of generating improved MHC proteins. Libraries of mutant MHC and mutant peptide-MHC complexes can be screened for higher surface levels in order to identify variants that exhibited improved properties, including enhanced stability. For the first time, this system allows the directed evolution of MHC molecules that represent novel agents for various diagnostic and therapeutic applications. These agents could be used for treatment or prevention of cancer, infectious diseases (e.g., virus infections), and autoimmune diseases (e.g., multiple sclerosis, type I diabetes, rheumatoid arthritis).

A number of strategies have been used or proposed to suppress autoimmune diseases, most notably drugs, such as cyclophosphamide, cyclosporin A, methotrexate, and Imuran (azathioprine). Steroid compounds, such as prednisone and methylprednisolone, are also employed in many instances. These drugs have limited long term efficacy against both cell- and antibody-mediated autoimmune diseases. Use of such drugs is limited by virtue of their toxic side effects that include “global” immunosuppression. Prolonged treatment with these drugs inhibits the normal protective immune response to pathogenic microorganisms, thereby increasing the risk of infections. A further drawback is that immune-mediated elimination of aberrant cells is impaired and there is, thus, an increased risk that malignancies will develop in patients receiving prolonged global immunosuppression.

The self substances, or autoantigens, which are the targets of autoimmune responses are most often protein products unique to the targeted cells (e.g., hormones such as insulin dependent diabetes mellitus, IDDM); particular enzymes unique to the specialized function of targeted cells (e.g., glutamic acid decarboxylase or GAD in IDDM, or 21 hydroxylase in Addison's disease); specialized cell-specific receptor molecules (e.g. the thyroid stimulating hormone (TSH) receptor in Graves' disease or acetylcholine receptors in the neuromuscular junctions in myasthenia gravis); and/or structural constituents of the targeted cells or tissues (e.g., beta cell sialo-glycoconjugate in IDDM). Prior to the current invention, immunization with autoantigens has been used as a means to induce autoimmune disease in experimental animals. For example, the administration of myelin basic protein (MBP) has been used as a means to induce experimental allergic encephalomyelitis (EAE, a model for MS) in mice. Additional treatments and prophylaxes are needed.

There is a long felt need in the art for Class II MHC proteins and Class II MHC/peptide complexes with improved stability and/or with improved T cell regulatory properties. Such improved Class II MHC proteins or complexes are useful in acting as antagonists of T cells that participate in the inappropriate removal of target cells or tissue. The improved Class II MHC proteins and complexes of the present invention are also improved for use as research tools in view of their improved stabilities. There is also an urgent need for prophylactic treatments to prevent serious autoimmune diseases such as Type I diabetes.

### SUMMARY OF THE INVENTION

The invention provides a combinatorial library of class II MHC proteins displayed on the surfaces of recombinant host cells, for example, yeast cells, desirably, Saccharomyces cerevisiae. From such a library can be isolated mutant MHC proteins that exhibit greater affinity for a ligand or a ligand peptide than the wild type Class II protein and/or Class II MHC proteins and Class II MHC/peptide complexes that are improved in biochemical stability over the corresponding wild type proteins and complexes.

Suitable labels allowing for use of a stable peptide-Class II MHC chimeric protein complex, especially a mutant Class II MHC protein or peptide-MHC complex having improved stability and/or improved binding, directly or indirectly, include but are not limited to fluorescent compounds, chemiluminescent compounds, radioisotopes, chromophores, and others. The labeled protein or complex of the present invention, where it specifically binds to a peptide of interest with high affinity and specificity, can be used in diagnostic tests to the particular type of autoimmune disease by virtue of the specific binding of the peptide-MHC Class II complex to a specific T cell receptor protein, and it can be used in the body in imaging tests to locate and/or estimate extent of autoimmune damage in progress, or it can be used as an antagonist or drug to eliminate T cells that cause autoimmune damage, potential or in progress.

### BRIEF DESCRIPTION OF THE DRAWINGS

FIG. 1 provides a ribbon diagram corresponding to the crystal structure of a class II major histocompatibility protein (the non-IDDM linked allele, IA^{d}) with highlighted amino acids of particular interest. The crystal structure of the non-IDDM linked murine allele, IA^{d}, is shown, displaying the influenza HA₁₂₆₋₁₃₈ peptide (hatched) (PDB code 2IAD; Scott et al., 1998). The IA^{d} β-chain and α-chain are highlighted in black and open shapes, respectively. IA^{g7} shares the same α-chain as IA^{d}, but differs by 17 residues in the β-chain including β56H and β57D shown in blue. β56H and β57S residues of IA^{g7} prevent a salt-bridge with α76R, perhaps contributing to its instability.

FIG. 2 provides diagrams for the genetic engineering of two single-chain Class II MHC constructs cloned into the yeast vector pCT302. The thrombin cleavage site allows the production of single chain, soluble Class II MHC protein complex (top diagram). In the alternative, the IA^{g7} construct allows for the insertion of a DNA fragment encoding a peptide ligand of the IA^{g7} molecule upstream of that region (bottom diagram). The GAD65 and insulin B9-23 peptides are important in the autoimmune destruction of insulin producing cells in the development of Type I Diabetes. The BDC2.5 (A) peptide is associated with IDDM as well.

FIG. 3 shows the results of flow cytometric analyses of various scIAI^{g7} constructions. The recombinant yeast cells expressing the scIA^{g7} were prepared, washed and incubated anti-HA mAb 12CA5 (Boehringer Mannheim, Indianapolis, Ind.), anti-c-myc mAb 9E10 (1:50 dilution of raw ascites fluid; Berkeley Antibody Co., Richmond, Calif.), or 10 μg/ml anti-IA^{g7} mAb 10.216 purified from hybridoma supernatant. Cells were then incubated with FITC-labeled F(ab′)₂ goat anti-mouse IgG (1:50) (Kirkegaard and Perry Labs, Inc., Gaithersburg, Md.), and labeled cells were analyzed by flow cytometry. As observed in the flow histograms, the Aga-2-IA^{g7} fusion in single chain format (Aga-2-HA-β-chain-linker-α-chain-c-myc) was not detectable on the yeast surface, which is consistent with the instability of the class II product. The Aga-2-IA^{g7} fusion with 3 peptides (GAD65 [78-96], B9-23 [Insulin], BDC2.5 [alanine stabilized variant]) linked at the IA^{g7} amino terminus α-chain exhibited low or undetectable levels of expression.

FIG. 4 summarizes the production of mutagenic libraries in order to generate stabilized MHC Class II, IA^{g7}. The random mutagenesis strategy employed the use of error-prone PCR with Primers 1 and 4 as described hereinbelow, and yeast homologous recombination to generate mutagenic libraries. Alternatively, a directed mutagenesis strategy can be employed, where degenerate Primers 2 and 3 and flanking Primers 1 and 4 are used in PCR reactions also as described hereinbelow. Isolated clones from each mutagenic strategy were rescued and sequenced to verify mutagenesis. Approximately 4-7 nucleotide errors were incorporated per 1000 base pairs in the random scIA^{g7} libraries.

FIG. 5 provides the result of sorting yeast homologous recombination mutagenic libraries (GAD65 [78-96] scIA^{g7}). Six random mutagenic scIA^{g7} yeast libraries were constructed with 10⁵-10⁶ independent transformants. Two scIA^{g7}β56β57 directed mutagenic libraries were constructed with 10⁴-10⁵ total independent transformants. The mutagenic libraries were then screened using a Cytomation MoFlo sorter (Cytomation, Fort Collins, Colo.) to isolate stabilized Aga-2-IA^{g7} fusions.

FIG. 6 shows the results of sorting libraries generated by random mutagenesis. GAD65(78-96), scIA^{g7} and B9-23 scIA^{g7} yeast libraries were incubated with 25 μl anti-IA^{g7} mAb 10.216 (10 μg/ml), washed with buffer (PBS/0.5% BSA), and incubated with FITC-labeled F(ab′)₂ goat anti-mouse IgG (1:50). After washing, samples were sorted in purification mode (coincident negative cells rejected) on a Cytomation MoFlo sorter with an event rate of ˜50,000 cells. A total of 2×10⁷ cells were examined during the first sorting round, collecting ˜1% of the population. Collected cells were re-grown at 30° C. in selective glucose medium for ˜18-20 h, and scIA^{g7} surface expression was induced at 20° C. in selective galactose medium. After three more rounds of sorting with anti-IA^{g7} mAb 10.216, the sorted libraries were incubated with 25 μl anti-c-myc mAb 9E 10 (1:50), washed with buffer (PBS/0.5% BSA), incubated with FITC-labeled F(ab′)₂ goat anti-mouse IgG, and sorted, collecting the top 0.25% of the population. The collected cells were plated on selective glucose medium to isolate individual clones. Clones were further examined using flow cytometry by staining with anti-IA^{g7} mAb 10.216 (FIG. 6B) and anti-c-myc ImAb 9E10 (FIG. 6A) followed by FITC-labeled F(ab′)₂ goat anti-mouse IgG. Plasmids from sorted scIA^{g7} yeast cells were rescued with a Zymoprep Miniprep kit (Zymo Research, Orange, Calif.). Rescued plasmid DNA was then transformed into E. coli DH10B competent cells by electroporation. Transformants were plated on LB plates supplemented with 100 μg/ml ampicillin. Sequencing was performed using scIA^{g7} flanking primers splice 4/L and T7 promoter, and a scIA^{g7} β-chain specific primer, scIA^{g7} α/βLNK (5′-CCA GGA CAG AGG CCC TCA AC-3′, SEQ ID NO:1), using fluorescence automated sequencing. Mutations in Mut 8 include GB 13A, Sβ57L, Wα43S and Vα139D.

FIGS. 7A-7B show the results of sorting exemplary GAD65 and B9-23scIA^{g7} error-prone library or yeast cells expressing the wild type B9-23 scIA^{g7} cell surface proteins with either anti-c-myc or anti-IA^{g7} antibodies. Residues differing from the wild type MHC Class II protein are shown at the bottom of the figure.

FIG. 8 shows sequences of clones isolated by sorting from GAD65(78-96) scIA^{g7} and B9-23 scIA^{g7} error-prone PCR libraries. The scIA^{g7} wild type amino acid sequence and residue numbers are shown with corresponding residue mutations of GAD65(78-96) scIA^{g7} and B9-23 scIA^{g7} error-prone clones. Multiple independent mutations were observed in both the scIA^{g7} β-chain and scIA^{g7} α-chain. This suggests that at least one of each of these mutations is linked to the increased stability of GAD65(78-96) scIA^{g7} and B9-23 scIA^{g7} mutants.

FIGS. 9A-9C show the results of successive sorts of the GAD65 scIA^{g7}β5657 library generated by directed mutagenesis. The GAD65 scIA^{g7}β5657 library was incubated with 25 μl anti-IA^{g7} mAb 10.216 (10 μg/ml), washed with buffer (PBS/0.5% BSA), and incubated with FITC-labeled F(ab′)₂ goat anti-mouse IgG (1:50). After washing, samples were sorted in purification mode using a Cytomation MoFlo sorter. A total of 2×10⁷ cells were examined during the first sorting round, collecting ˜0.25% of the population. Collected cells were re-grown at 30° C. in selective glucose medium for ˜18-20 h and scIA^{g7} surface expression was induced at 20° C. in selective galactose medium. Following the second sort with anti-IA^{g7} mAb 10.216, the sorted library was incubated with 25 μl anti-c-myc mAb 9E10 (1:50), washed with buffer (PBS/0.5% BSA), incubated with FITC-labeled F(ab′)₂ goat anti-mouse IgG, and sorted, again collecting the top 0.25% of the population. Sorted clones were further analyzed by flow cytometry.

FIGS. 10A-10B show the results obtained with an exemplary clone isolated by sorting the GAD65 scIA^{g7}β5657 library. Results are shown for sorts with an anti-c-myc antibody and with anti-IA^{g7} antibody.

FIG. 11 shows the results of rapid (one day) sequential sorting of the randomly mutated BDC2.5 sc IA^{g7}β5657 library. The BDC2.5 scIA^{g7}β5657 yeast library was stained with 12.5 μl anti-IA^{g7} and biotin-labeled anti-c-myc antibody, incubated with FITC-labeled F(ab′)₂ goat anti-mouse, γ_{2b} chain specific, IgG_{2b} and streptavidin-phycoerythrin (SA:PE) conjugate. After washing, samples were sorted in purification mode (coincident negative cells rejected) using a fluorescence activated cell sorter. About 1% of the total cells examined in the first sort were collected. The collected cells were sequentially sorted twice more on the same day, collecting the top 1% of the population each time. The cells collected from the third sort were plated and then further examined by flow cytometry.

FIG. 12 provides a summary of clones isolated by fluorescence activated cell sorting from a BDC2.5 scIA^{g7}β5657 mutant library. Binding levels are shown as a % positive population shift to anti-c-myc mAb and anti-IA^{g7} mAb from BDC2.5 scIA^{g7}β5657 clones isolated from the final sequential sort. Nine mutants in addition to BDC2.5 scIA9 wild type yeast, B9-23 scIA^{g7} Mut8/yeast (anti-IA^{g7} mAb positive control), 7Msc4F10/yeast (anti-c-myc mAb positive control), and EBY100 (negative control) yeast were induced in galactose medium overnight at 30° C. Cells were analyzed by flow cytometry after staining with anti-c-myc mAb (black, stippled bars), or stained with anti-IA^{g7} mAb (cross-hatched bars) followed by FITC-labeled F(ab′)₂ goat anti-mouse IgG. Mutants isolated yielded higher surface level binding to anti-c-myc and anti-IA^{g7} antibodies than its BDC2.5 scIA^{g7} wild type counterpart. BDC2.5 scIA^{g7}β5657 mutants were sequenced and contained the consensus motifs of E/G₅₆ and L/M₅₇.

FIG. 13 shows the binding peptide B-1040-63 to IA^{g7} transfected L cells. See Example 2 for experimental details.

FIG. 14A provides a diagram of a scIA^{g7} β₁α₁ fusion, and

FIG. 14B provides a diagram of a peptide scIA^{g7} β₁α₁ fusion.

### DETAILED DESCRIPTION OF THE INVENTION

The role of proteins encoded by the major histocompatibility complex (called MHC proteins) has now been known for over twenty years. MHC proteins are expressed by every individual and function as “antigen-presenting” molecules. That is, each MHC protein can bind to a variety of different small peptides (8 to 20 amino acids in length) that are derived from proteins present inside a cell. MHC proteins present both self-peptides (i.e., derived from an individual's own endogenous proteins) and foreign peptides (i.e., derived from a foreign agent such as a virus). Once a peptide is bound to the MHC protein, the entire peptide-MHC complex (pMHC) is expressed on the surface of the cell. If the peptide is foreign, a T lymphocyte (T cell) can potentially recognize the complex, and the resultant interaction of the T cell receptor (TCR) and the pMHC can result in T cell activation. T cell activation can lead to recruitment of other immune cells and a corresponding inflammatory reaction. Such inflammatory reactions are beneficial if the pMHC target antigen is in fact derived from an infectious agent or from a neoplastic cell (i.e., cancer). However, such inflammatory reactions can be very detrimental if the pMHC target antigen is derived from self tissue, as the reactions can lead to severe autoimmune disease, where an individual's immune system attacks normal tissue. Such is the case when a patient's lymphocytes attack the islet cells of the pancreas (type I diabetes), the nervous system (multiple sclerosis), or joint-derived components (rheumatoid arthritis).

The central role of pMHC complexes in these phenomena has been established by thousands of published studies that include genetic linkages of diseases to the human MHC locus (HLA). It has now also been established that it is possible to use appropriately characterized peptide-MHC molecules as either agonists of an immune response (e.g., in cancer and infectious diseases) or as antagonists of responses (e.g., in autoimmune responses). While several approaches have been taken to produce such pMHC complexes in soluble forms for these purposes and for biochemical/structural studies, it has not been possible to use current methods of in vitro directed evolution to improve the stability or antigenicity of the pMHC complex. This is because the pMHC complex is normally a membrane-associated complex composed of multiple different subunits (heavy chain, beta-2-microglobulin, and peptide in the case of a class I MHC, and α-chain, β-chain, and peptide in the case of class II MHC) and such proteins are typically not amenable to the current methods of directed evolution (primarily phage display). The present invention shows that a display system for directed evolution can be used to express properly folded class I and class II MHC proteins on the surface of yeast. The displayed peptide-MHC complexes can be used to directly activate T cells, for treatment or in order to identify/screen for pMHC antigens. In addition, mutated libraries of the pMHC proteins can be created and used for selection by flow sorting of stabilized pMHC variants. The stabilized variants could be identified because they were expressed at higher levels on the yeast surface and could therefore be easily identified by using a fluorescent-labeled probe for the pMHC construct, combined with high-throughput flow cytometric sorting of such cells.

The MHC Class II proteins have been associated with susceptibility and resistance to autoimmune disorders. Insulin dependent diabetes mellitus (IDDM) has been linked to certain murine I-A alleles and human HLA-DQ homologues. In all cases where an MHC Class II allele is associated with IDDM, there is a lack of D57 in the β chain of the MHC protein, whereas the D57 residue is present in the non-IDDM-linked alleles. The T_{H}1 inflammatory response which results in destruction of islet cells in IDDM is believed to result from poor central tolerance or promiscuous peptide binding of disease-linked MHC proteins.

The non-obese (NOD) mouse is the generally accepted model for the study of IDDM. NOD mice spontaneously develop IDDM early in life due to the disease-associated MHC II haplotype I-A^{g7}. I-A^{g7} shares the same α chain as I-A^{d}, but it differs by 17 residues in the β chain, including the β56H and β57S, which confer the I-A^{g7} diabetogenic character. Replacement of the I-A^{g7} β56H and β57S residues drastically reduces the incidence of diabetes in NOD mice. Populations of I-A^{g7} have been shown to be susceptible to sodium dodecyl sulfate denaturation, and they have relatively weak peptide binding, perhaps allowing the T cells to escape negative selection (purging of self-reactive clones) in the thymus.

With the goal of isolating mutant forms of Class II MHC proteins and protein/peptide complexes, a single chain fusion protein has been expressed through the use of genetic engineering technology. See, e.g., WO 99/36569, incorporated by reference herein, for a discussion of yeast surface display technology and vectors. See also U.S. Provisional Application No. 60/254,495 filed Dec. 8, 2000, also incorporated by reference herein. As specifically exemplified herein, the Class II protein is expressed as a single chain protein of the format AGA2-HA-β chain-linker-α chain-c-myc. The wild type fusion protein is not detected on the yeast cell surface, thus reflecting the instability of the wild type Class II MHC protein.

Class II I-A^{g7} has been cloned as an AGA2 fusion with 3 peptides (GAD65, insulin B9-23 and BDC 2.5(alanine stabilized variant of the BDC2.5 peptide mimic, GKKVAAPVWIRMG, SEQ ID NO:21) linked at the amino terminus of the fusion protein. Low or undetectable expression levels result.

To create stabilized I-A^{g7} variants, eight different mutational libraries were produced by error prone polymerase chain reaction (PCR) to produce random mutations, and oligonucleotide site directed mutagenesis of residues 56 and 57 was carried out using homologous recombination after co-electroporation of the mutated coding sequence-containing nucleic acid molecules and linearized vector (pCT302 or pYD1, available from Invitrogen, Carlsbad, Calif.). Sorting of the randomly mutated GAD65 and B9-23 libraries with anti-c-myc and anti-I-A^{g7} antibodies yielded many mutants with higher surface levels of the fusion protein, indicating increased stability of the molecule. Sorting of the BCD2.5 β56/β57 library with anti-c-myc and anti-I-A^{g7} antibodies also yielded many mutants with higher surface levels of the fusion protein, indicating increased stability of the molecule. These mutants showed consensus motifs of E/G₅₆ and L/M₅₇.

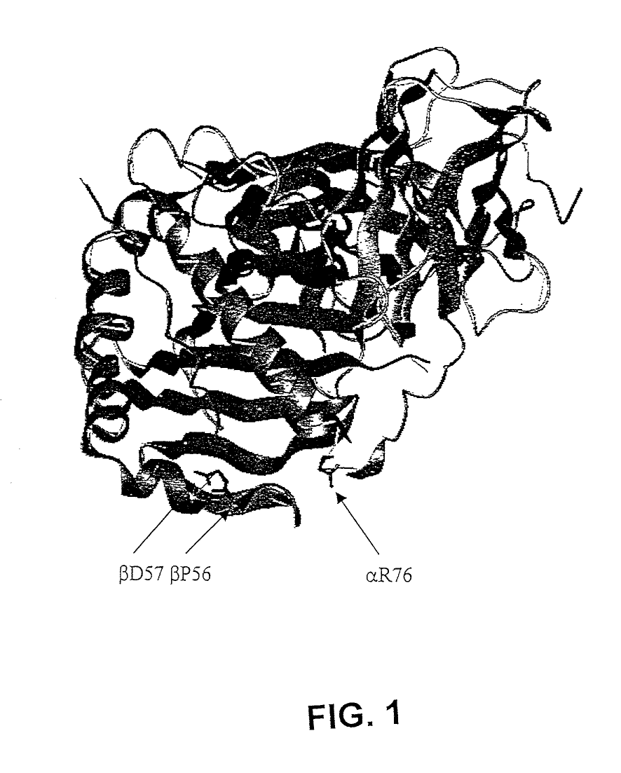

The crystal structure of non-IDDM linked allele, I-A^{d} is shown in FIG. 1. Insulin-dependent diabetes mellitus (IDDM) is associated with certain murine I-A alleles, such as IA^{g7}, and human HLA-DQ homologues. The crystal structure of a non-IDDM linked murine allele, IA^{d}, is shown, displaying the influenza HA₁₂₆₋₁₃₈ peptide (PDB code 2IAD; Scott et al., 1998). The IA^{d} β-chain and α-chain are also shown. IA^{g7} shares the same α-chain as IA^{d}, but differs by 17 residues in the β-chain, including β56H and β57D. β56H and β57S residues of IA^{g7} prevent salt-bridge formation with α76R, perhaps contributing to its instability.

The present invention allows the creation and isolation of stabilized variants of Class II peptide-MHC complexes. Toward this end, we have displayed single-chain peptide/Class II MHC complexes on the surface of yeast cells, and we have isolated stabilized variants of the I-A^{g7} molecule in association with each of three peptides of interest.

WO 99/36569 describes the yeast display technology in general terms. The MHC protein of interest is displayed on the yeast cell surface via a disulfide linkage through the AGA2 portion of the fusion protein comprising the MHC component. AGA2 is a mating adhesion receptor which is naturally bound to the cell surface in disulfide linkage to the AGA1 protein. The HA and the c-myc portions of the displayed fusion protein serve as epitope tags and can be used in normalizing the fluorescent peptide binding data. Each recombinant yeast cell displays about 50,000 copies of the surface bound fusion protein (if stable) on its surface. A fluorescent antibody or peptide ligand specific for the MHC protein of interest is added, and the cells are sorted using flow cytometry. Those MHC fusion proteins of increased stability exhibit stronger binding of the fluorescent ligand, and these cells are selected during the cell sorting procedure.

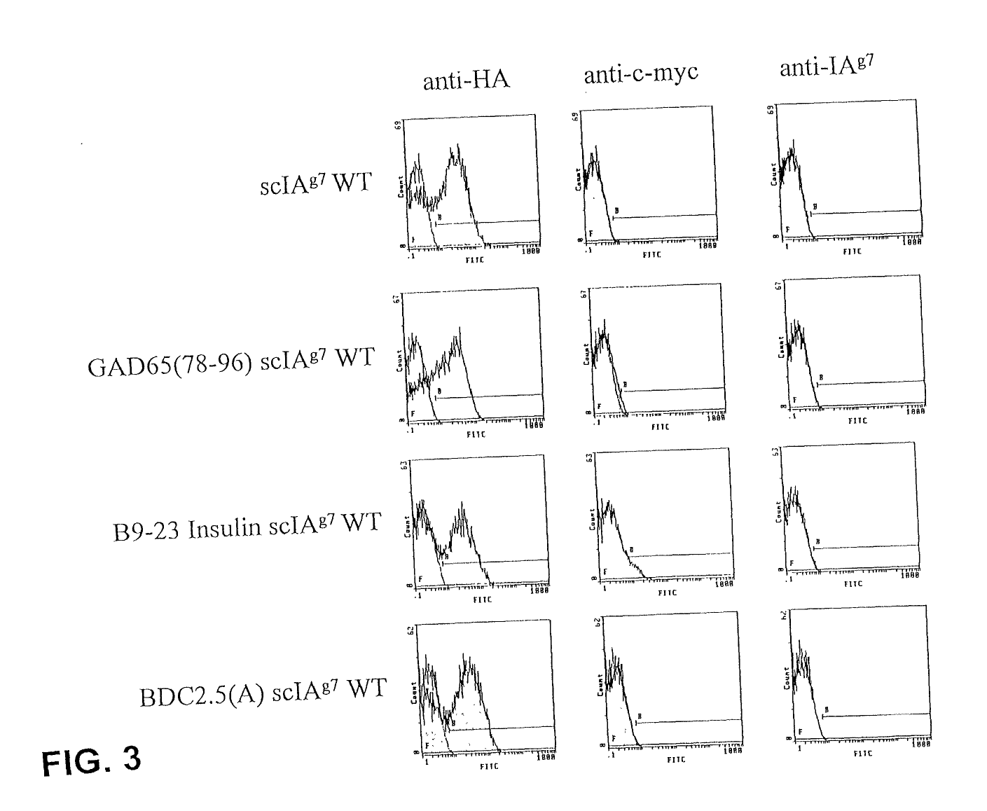

FIG. 3 illustrates diagrammatically the pCT302 yeast surface display vector that contains a sequence encoding AGA2/HA-Class II MHC-c-myc fusion protein. This fusion protein coding sequence is expressed in yeast under the regulatory control of the inducible GAL 1-10 promoter.

The yeast display system was exploited to produce a random mutagenized library from which stabilized mutant Class II MHC sequences were isolated. Constructs encoding the fusion proteins were mutagenized randomly using error prone PCR (0.16 Mn:Mg molar ratio). A homologous recombination scheme was employed to create the libraries.

In order to provide a clear and consistent understanding of the specification and claims, including the scope to be given to such terms, the following definitions are provided.

A coding sequence is the part of a gene or cDNA which codes for the amino acid sequence of a protein, or for a functional RNA such as a tRNA or rRNA.

Complement or complementary sequence means a sequence of nucleotides which forms a hydrogen-bonded duplex with another sequence ofnucleotides according to Watson-Crick base-pairing rules. For example, the complementary base sequence for 5′-AAGGCT-3′ is 3′-TTCCGA-5′.

Downstream means on the 3′ side of any site in DNA or RNA.

Expression refers to the transcription of a gene into structural RNA (rRNA, tRNA) or messenger RNA (mRNA) and subsequent translation of a mRNA into a protein.

An amino acid sequence that is functionally equivalent to a specifically exemplified class II MHC protein sequence is an amino acid sequence that has been modified by single or multiple amino acid substitutions, by addition and/or deletion of amino acids, or where one or more amino acids have been chemically modified, but which nevertheless retains the binding specificity and high affinity binding activity of a cell-bound or a soluble MHC protein of the present invention. Functionally equivalent nucleotide sequences are those that encode polypeptides having substantially the same biological activity as a specifically exemplified cell-bound or soluble MHC protein. In the context of the present invention, a soluble MHC protein lacks the portions of a native cell-bound MHC and is stable in solution (i.e., it does not generally aggregate in solution when handled as described herein and under standard conditions for protein solutions).

Two nucleic acid sequences are heterologous to one another if the sequences are derived from separate organisms, whether or not such organisms are of different species, as long as the sequences do not naturally occur together in the same arrangement in the same organism.

Homology refers to the extent of identity between two nucleotide or amino acid sequences.

Isolated means altered by the hand of man from the natural state. If an “isolated” composition or substance occurs in nature, it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

A linker region is an amino acid sequence that operably links two functional or structural domains of a protein.

A nucleic acid construct is a nucleic acid molecule which is isolated from a naturally occurring gene or which has been modified to contain segments of nucleic acid which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

Two DNA sequences are operably linked if the nature of the linkage does not interfere with the ability of the sequences to effect their normal functions relative to each other. For instance, a promoter region would be operably linked to a coding sequence if the promoter were capable of effecting transcription of that coding sequence.

A polypeptide is a linear polymer of amino acids that are linked by peptide bonds.

Promoter means a cis-acting DNA sequence, generally 80-120 base pairs long and located upstream of the initiation site of a gene, to which RNA polymerase binds and initiates correct transcription. There can be associated additional transcription regulatory sequences which provide on/off regulation of transcription and/or which enhance (increase) expression of the downstream coding sequence.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site. Alternatively, a recombinant DNA molecule can result from homologous recombination after co-transformation (or co-electroporation) of two DNA molecules sharing at least limited sequence identity.

Transformation means the directed modification of the genome of a cell by the external application of purified recombinant DNA from another cell of different genotype, leading to its uptake and possibly its integration into the subject cell's genome. In bacteria, the recombinant DNA is not typically integrated into the bacterial chromosome, but instead replicates autonomously as a plasmid.

Upstream means on the 5′ side of any site in DNA or RNA.

A vector is a nucleic acid molecule that is able to replicate autonomously in a host cell and can accept foreign DNA. A vector carries at least one origin of replication functional in at least one type of cell, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include plasmid vectors and phage vectors. There can be more than one origin of replication to allow for replication and maintenance in more than one type of cell (e.g., separate origins of replication functional in yeast and Escherichia coli, respectively).

Autoimmune destruction of tissue, in the present context, means that the immune system of an individual or animal has inappropriately targeted that tissue for killing. One important example is the autoimmune destruction of the insulin producing islet cells of the pancreas, which results in insulin dependent diabetes mellitus (Type I diabetes). Another example is the destruction of myelin surrounding nerve fibers in multiple sclerosis. In the case of Type I diabetes, two peptide antigens have been identified as important in the autoimmune response. These include the “B9-23” peptide of insulin (encompassing amino acids 9-23 of the B chain of human insulin) and a peptide derived from the 65 kDa glutamate decarboxylase protein (GAD65; amino acids 78-96). See Table 2 for the amino acid sequences of these peptides.

The role of proteins encoded by the major histocompatibility complex (MHC proteins) have been known for over twenty years. MHC proteins are expressed by every individual, and they function as antigen-presenting molecules. Each MHC protein can bind to a variety of different small peptides (of 8 to 20 amino acids). T cells recognize a foreign peptide bound to the MHC product through the αβ heterodimeric T cell receptor (TCR). The TCR repertoire has extensive diversity created by the same gene rearrangement mechanisms used in antibody heavy and light chain genes [Tonegawa, S. (1988) Biosci. Rep. 8:3-26]. Most of the diversity is generated at the junctions of variable (V) and joining (J) (or diversity, D) regions that encode the complementarity determining region 3 (CDR3) of the α and β chains [Davis and Bjorkman (1988) Nature 334:395-402]. However, TCRs do not undergo somatic point mutations as do antibodies and, perhaps not coincidentally. The serial-triggering [Valitutti et al. (1995) Nature 375:148-151] and kinetic proofreading [Rabinowitz et al. (1996) Proc. Natl. Acad. Sci. USA 93:1401-1405] models of T cell activation both suggest that longer off-rates (associated with higher affinity) are detrimental to the signaling process. It is also possible that higher affinity TCRs do not maintain the peptide specificity required for T cell responses. For example, peptides bound within the MFHC groove display limited accessible surface [Bjorkman, P. J. (1997) Cell 89:167-170], which may in turn limit the amount of energy that can be generated in the interaction. On the other hand, raising the affinity of a TCR by directing the energy toward the MHC helices leads to thymic depletion during negative selection [Bevan, M. J. (1997) Immunity 7:175-178].

In summary, we have shown that Class II MHC proteins and protein-peptide complexes can be engineered to yield proteins and complexes of increased biochemical stability. The stabilized Class II MHC derivatives are useful in diagnosis or study of certain autoimmune diseases, and they are useful as antagonists of T cell-mediated autoimmune destruction of target tissues, for example, the destruction of insulin producing islet cells of the pancreas in the development of insulin dependent diabetes mellitus.

It will be appreciated by those of skill in the art that, due to the degeneracy of the genetic code, numerous functionally equivalent nucleotide sequences encode the same amino acid sequence.

Additionally, those of skill in the art, through standard mutagenesis techniques, in conjunction with the antigen-finding activity assays described herein, can obtain altered class II MHC sequences and test them for the expression of polypeptides having particular binding activity or improved biochemical stability. Useful mutagenesis techniques known in the art include, without limitation, oligonucleotide-directed mutagenesis, region-specific mutagenesis, linker-scanning mutagenesis, and site-directed mutagenesis by PCR [see e.g. Sambrook et al. (1989) and Ausubel et al. (1999)].

In obtaining variant MHC Class II coding sequences, those of ordinary skill in the art will recognize that MHC-derived proteins can be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50° C., 6× SSC (sodium chloride/sodium citrate buffer) and 0.1% SDS (sodium dodecyl sulfate) indicate about 60-70% homology, hybridization and wash conditions of 50-65° C., 1× SSC and 0.1% SDS indicate about 82-97% homology, and hybridization and wash conditions of 52° C., 0.1× SSC and 0.1% SDS indicate about 99-100% homology. A wide range of computer programs for comparing nucleotide and amino acid sequences (and measuring the degree of homology) are also available, and a list providing sources of both commercially available and free software is found in Ausubel et al. (1999). Readily available sequence comparison and multiple sequence alignment algorithms are, respectively, the Basic Local Alignment Search Tool (BLAST) [Altschul et al., (1997) Nucl. Acids Res. 25:3389-3402] and ClustalW programs. BLAST is available on the Internet at http://www.ncbi.nlm.nih.gov and a version of ClustalW is available at http://www2.ebi.ac.uk.

Industrial strains of microorganisms (e.g., Aspergillus niger, Aspergillus ficuum, Aspergillus awamori, Aspergillus oryzae, Trichoderma reesei, Mucor miehei, Kluyveromyces lactis, Pichiapastoris, Saccharomyces cerevisiae, Escherichia coli, Bacillus subtilis or Bacillus licheniformis) or plant species (e.g., canola, soybean, corn, potato, barley, rye, wheat) may be used as host cells for the recombinant production of the stabilized mutant Class II MHC proteins of the present invention. As the first step in the heterologous expression of a high affinity MHC protein or soluble protein, an expression construct is assembled to include the MHC or soluble MHC coding sequence and control sequences such as promoters, enhancers and terminators. Other sequences such as signal sequences and selectable markers may also be included. To achieve extracellular expression of a soluble MHC polypeptide, the expression construct may include a secretory signal sequence. The signal sequence is not included on the expression construct if cytoplasmic expression is desired. The promoter and signal sequence are functional in the host cell and provide for expression and secretion of the MHC or soluble MHC protein. Transcriptional terminators are included to ensure efficient transcription. Ancillary sequences enhancing expression or protein purification may also be included in the expression construct.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such tac are often employed in order to dramatically increase the level of protein expression E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the TCR coding sequence may be used. Alternatively, a signal sequence which has been selected or designed for efficient secretion and processing in the expression host may also be used. For example, suitable signal sequence/host cell pairs include the B. subtilis sacB signal sequence for secretion in B. subtilis, and the Saccharomyces cerevisiae α-mating factor or P. pastoris acid phosphatase phoI signal sequences for P. pastoris secretion. The signal sequence may be joined directly through the sequence encoding the signal peptidase cleavage site to the protein coding sequence, or through a short nucleotide bridge consisting of usually fewer than ten codons, where the bridge ensures correct reading frame of the downstream TCR sequence.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His⁻ S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct is assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Numerous cloning vectors suitable for construction of the expression construct are known in the art (λZAP and pBLUESCRIPT SK-1, Stratagene, La Jolla, Calif.; pET, Novagen Inc., Madison, Wis.; cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (Ausubel et al., 1999; Sambrook et al., 1989). The gene transfer system selected depends upon the host cells and vector systems used.

For instance, the expression construct can be introduced into S. cerevisiae cells by protoplast transformation or electroporation. Electroporation of S. cerevisiae is readily accomplished, and yields transformation efficiencies comparable to spheroplast transformation. Co-electroporation of a linearized vector and a linear DNA molecule of interest having regions of homology to the vector at each end results in homologous recombination within the yeast cell, thus circumventing the need for ligation in vitro prior to transformation of the yeast cells.

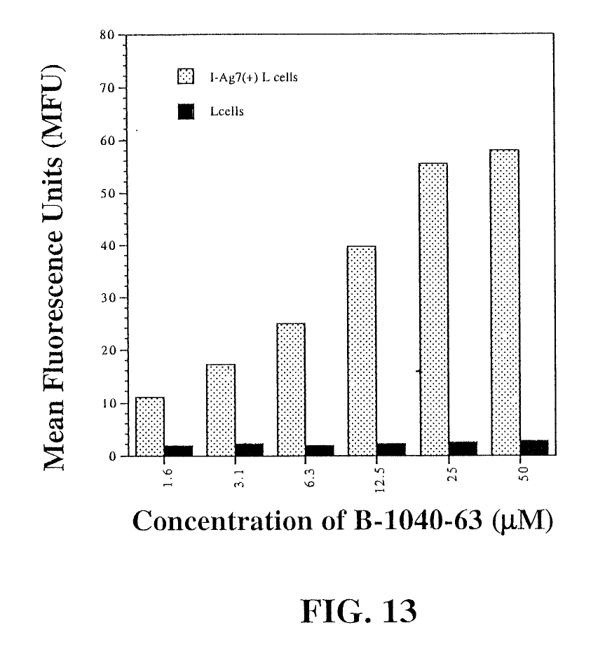

Now that stabilized IA^{g7} mutants have been isolated by the process of directed evolution and yeast display technology as described herein, those IA^{g7} mutants that are capable of binding peptides are identified. To perform these studies we required a labeled-IA^{g7}-binding peptide that could be used as a probe for further selection of IA^{g7} mutants on yeast. We have used a biotinylated peptide called B-1040-63 (Judkowski et al., 2001) for this purpose. This biotinylated peptide was shown to bind specifically to L cells that were transfected with IA^{g7} gene, but it does not bind to untransfected L cells (FIG. 13). This biotinylated peptide is used to select peptide-binding mutants from the yeast library that expresses stabilized IA^{g7} mutants. Such stabilized peptide-IA^{g7} complexes are then used to confirm their ability to regulate T cell activity.

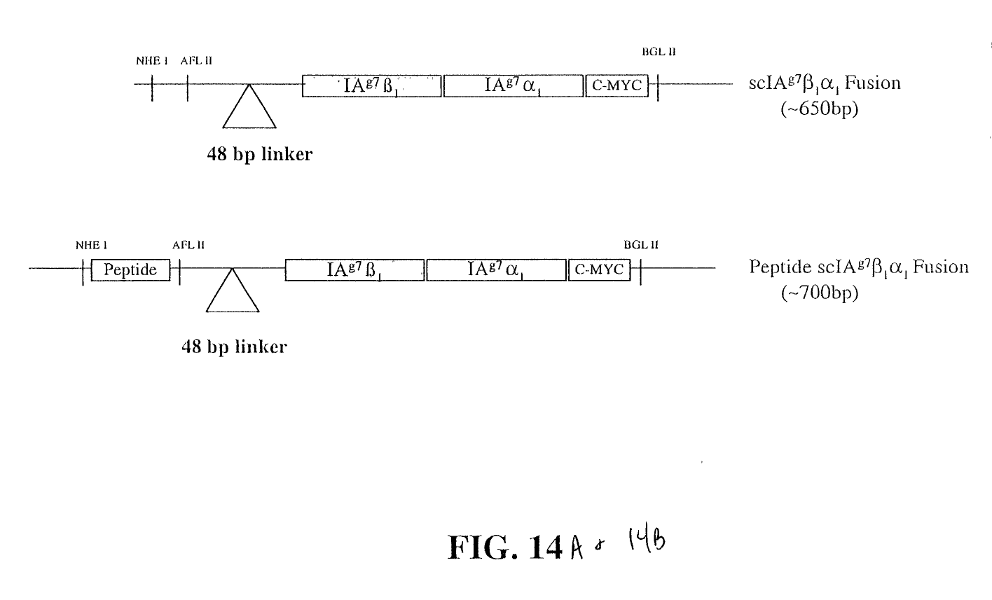

The second goal has been to explore whether mutants of even smaller class II peptide binding modules can be produced by expressing only the N-terminal (α₁ and β₁) domains of the IA^{g7} molecule (Chang et al., 2001). The single-chain constructions shown in FIG. 14 have now been generated. Yeast libraries with mutated versions of these proteins are selected for anti-IA^{g7} and peptide binding in order to isolate even smaller agents that can be used to regulate T cells.

Monoclonal or polyclonal antibodies, preferably monoclonal, specifically reacting with an MHC protein at a site other than the ligand binding site may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2ded., Academic Press, New York; and Ausubel et al. (1999) Current Protocols in Molecular Biology, John Wiley & Sons, Inc., New York.

Stable Class II MHC proteins in cell-bound or soluble form which are bound to a particular peptide as a complex are useful, for example, as diagnostic probes for screening biological samples (such as cells, tissue samples, biopsy material, bodily fluids and the like) for the presence of T cells displaying a T cell receptor protein specific for the peptide-MHC complex. In addition, they can serve as antagonists of T lymphocyte-mediated destruction of cells or tissue expressing the particular peptide. Frequently, the stable Class II MHC proteins are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. Additionally the MHC protein can be coupled to a ligand for a second binding molecules: for example, the MEC protein can be biotinylated. United States Patents describing the use of labels and/or toxic compounds to be covalently bound to the Class II MHC stabilized protein or complex include, but are not limited, to U.S. Pat. Nos. 3,817,837; 3,850,752; 3,927,193; 3,939,350; 3,996,345; 4,277,437; 4,275,149; 4,331,647; 4,348,376; 4,361,544; 4,468,457; 4,444,744; 4,640,561; 4,366,241; RE No. 35,500; U.S. Pat. Nos. 5,299,253; 5,101,827; 5,059,413. Labeled (detectable) MHC Class II proteins can be detected using a monitoring device or method appropriate to the label used. Fluorescence microscopy or fluorescence activated cell sorting can be used where the label is a fluorescent moiety, and where the label is a radionuclide, gamma counting, autoradiography or liquid scintillation counting, for example, can be used with the proviso that the method is appropriate to the sample being analyzed and the radionuclide used. In addition, there can be secondary detection molecules or particle employed where there is a detectable molecule or particle that recognized the proteins. The art knows useful compounds for diagnostic imaging in situ; see, e.g., U.S. Pat. No. 5,101,827 or 5,059,413. Radionuclides useful for therapy and/or imaging in vivo include ¹¹¹Indium, ⁹⁷Rubidium, ¹²⁵Iodine, ¹³¹Iodine, ¹²³Iodine, ⁶⁷Gallium, ⁹⁹Technetium. Toxins include diphtheria toxin, ricin and castor bean toxin, among others, with the proviso that once the MHC-toxin complex is bound to the cell, the toxic moiety is internalized so that it can exert its cytotoxic effect. Immunotoxin technology is well known to the art, and suitable toxic molecules include, without limitation, chemotherapeutic drugs such as vindesine, antifolates, e.g. methotrexate, cisplatin, mitomycin, anthrocyclines such as daunomycin, daunorubicin or adriamycin, and cytotoxic proteins such as ribosome inactivating proteins (e.g., diphtheria toxin, pokeweed antiviral protein, abrin, ricin, pseudomonas exotoxin A or their recombinant derivatives. See, generally, e.g., Olsnes and Pihl (1982) Pharmac. Ther. 25:355-381 and Monoclonal Antibodies for Cancer Detection and Therapy, Eds. Baldwin and Byers, pp. 159-179, Academic Press, 1985.

Stable, high affinity MHC proteins specific for a particular ligand, e.g., a particular peptide, protein or cell type, are useful in diagnosing animals, including humans, believed to be suffering from a disease associated with the particular pMHC. The MHC molecules of the present invention are useful for detecting T cells that are specific for essentially any antigen including, but not limited to, those associated with a neoplastic condition, an abnormal protein, or an infection or infestation with a bacterium, a fungus, a virus, a protozoan, a yeast, a nematode or other parasite. The proteins can also be used in the diagnosis of certain genetic disorders in which there is a stabilized MHC Class II abnormal protein produced. Exemplary applications for these stable, high affinity proteins is in the treatment of autoimmune diseases in which there is a known pMHC. Type I diabetes is relatively well characterized with respect to the autoantigens which attract immune destruction. Multiple sclerosis, celiac disease, inflammatory bowel disease, Crohn's disease and rheumatoid arthritis are additional candidate diseases for such application. Stabilized Class II MHC proteins with binding specificity for a particular peptide on the surface of cells or tissues which are improperly targeted for autoimmune destruction can serve as antagonists of the autoimmune destruction by competing for binding to the target cells by T cells or by directly inactivating the T cell. Such stabilized MHC proteins can be obtained by the methods described herein and subsequently used for screening for T cells that are specific for a particular ligand of interest.

The stabilized MHC compositions, especially as the stable mutant peptide-MHC chimeric protein or protein complex, can be formulated by any of the means known in the art. They can be typically prepared as injectables, especially for intravenous, intraperitoneal or synovial administration (with the route determined by the particular disease) or as formulations for intranasal or oral administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active ingredients are often mixed with excipients or carriers which are pharmaceutically acceptable and compatible with the active ingredient. Suitable excipients include but are not limited to water, saline, dextrose, glycerol, ethanol, or the like and combinations thereof The concentration of the MHC protein in injectable, aerosol or nasal formulations is usually in the range of 0.05 to 5 mg/ml. Similar dosages can be administered to other mucosal surfaces.

In addition, if desired, vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. Such additional formulations and modes of administration as are known in the art may also be used.

The stabilized high affinity MHC Class II proteins of the present invention and/or pMHC-binding fragments having primary structure similar (more than 90% identity) to the high affinity MHC proteins and which maintain the improved stability and/or the high affinity for the cognate ligand may be formulated into vaccines as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine. Alternatively, these high affinity MHC proteins can be used as antagonists of an interaction between endogenous MHC proteins of similar specificity and the cognate TCR cells.

MHC proteins for therapeutic use, e.g., those conjugated to cytotoxic compounds are administered in a manner compatible with the dosage formulation, and in such amount and manner as are prophylactically and/or therapeutically effective, according to what is known to the art. The quantity to be administered, which is generally in the range of about 100 to 20,000 μg of protein per dose, more generally in the range of about 1000 to 10,000 μg of protein per dose. Similar compositions can be administered in similar ways using labeled high affinity MHC proteins for use in imaging, for example, to detect T cells that are involved in an autoimmune attack and express the TCRs that are specific for the tissue that is the target of the autoimmune attack. Precise amounts of the active ingredient required to be administered may depend on the judgment of the physician or veterinarian and may be peculiar to each individual, but such a determination is well within the skill of such a practitioner.

The vaccine or other immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the retrovirus-like particles of the present invention are protected from infection by the cognate retrovirus.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

All references cited in the present application are incorporated by reference herein to supplement the disclosure and experimental procedures provided in the present Specification to the extent that there is no inconsistency with the present disclosure.

The following examples are provided for illustrative purposes, and are not intended to limit the scope of the invention as claimed herein. Any variations in the exemplified articles and/or methods which occur to the skilled artisan are intended to fall within the scope of the present invention.

### EXAMPLES

### Example 1

### Engineering of Single-Chain Class II MHC, IA^{g7}

To construct a recombinant vector for the expression of a single chain Class II MHC protein, the IA^{g7} β-chain was PCR amplified using the forward primer (5′-ATT GCA GCT AGC GGT GGA CCT AAG GGT GGC GGC GGT TCT TTA GTT CCA AGA GGT TCT GGT GGC GGT GGC TCT GGA GAC TCC GAA AGG CAT TT-3′, SEQ ID NO:2) incorporating Nhe1/AflII restriction sites and 16 AA linker upstream of the β-chain and reverse primer (5′-TCC GCC ACC TCC AGA ACC TCC TCC GCC CCT CCA CTC CAC AGT GAT GGG-3′, SEQ ID NO:3) containing the 9 AA linker downstream of the β-chain. The IA^{g7} α-chain was amplified with the forward primer (5′-GGC GGA GGA GGT TCT GGA GGT GGC GGA GAA GAC GAC ATT GAG GCC-3′, SEQ ID NO:28) that contained the same 9 amino acid linker upstream of the α-chain and reverse primer (5′-ATT TGC AGA TCT TTA TCA CAA GTC TTC TTC AGA AAT AAG CTT TTG TTC CCA GTG TTT CAG AAC CGG CTC-3′ SEQ ID NO:4) incorporating the c-myc epitope tag and BglII diagnostic site downstream of the α-chain. PCR sewing was then used to fuse IA^{g7} β-chain and α-chain PCR products through and additional amplification using the β-chain forward primer and the α-chain reverse primer.

The GAD65(78-96) scIA^{g7} construct was generated by PCR amplification of the scIA^{g7} fusion product using a forward primer (5′-ATT GCA GCT AGC AAA CCA TGT AAT TGT CCA AAA GGT GAT GTT AAT TAT GCT TTT TTG CAT GCT ACT GAT CTT AAG GGT GGC GGC GGT TCT TTA GTT CCA-3′, SEQ ID NO:5) that incorporated the GAD65(78-96) peptide between the Nhe1 and AflII restriction sites and the α-chain reverse primer. The generated scIA^{g7} and GAD65(78-96) scIA^{g7} constructs were digested with Nhe1 and BglII and ligated to Nhe1-BglII-digested yeast surface display vector pCT302 containing a nine-residue epitope tag (HA) and the AGA2 open reading frame downstream of the inducible GAL1 promoter (Boder and Wittrup, 1997). The ligation mixture was transformed into electro-competent E. coli DH10B (Gibco BRL/Invitrogen, Carlsbad, Calif.), and transformants were plated on LB plates supplemented with ampicillin at 100 μg/ml and grown overnight at 37° C.

Cassette ligations were used to generate B9-23 scIA^{g7} and BDC2.5(A) scIA^{g7} constructs. BDC2.5(A) peptide and B9-23insulin peptide sense (5′-CTA GCG GTA AAA AGG TTG CTG CAC CAG CTT GGG CTC GTA TGG GTC-3′, SEQ ID NO:6; 5′-CTA GCT CTC ATT TGG TTG AAG CTT TGT ATT TGG TTT GTG GTG AAA GAG GTC-3′, SEQ ID NO:7) and anti-sense (5′-TTA AGA CCC ATA CGA GCC CAA GCT GGT GCA GCA ACC TTT TTA CCG-3′, SEQ ID NO:8; 5′-TTA AGA CCT CTT TCA CCA CAA ACC AAA TAC AAA GCT TCA ACC AAA TGA GAG-3′, SEQ ID NO:9) 5′-phosphorylated oligonucleotides with Nhe1 and AflII restriction site overhangs were mixed in equimolar ratios. Peptide-specific forward and reverse primers were incubated at 100° C. for 1 minute, and allowed anneal at 25° C., generating the peptide cassettes. The cassettes were then ligated to Nhe1-AflII-digested GAD65(78-96) scIA^{g7}/pCT302 and transformed into E. coli as described previously. Plasmid DNA was transformed into the yeast strain EBY100 according to published methods (Geitz et al., 1995). Transformants are selected for tryptophan prototrophy.

### Example 2

### Flow Cytometric Analysis

FIG. 3 shows the results of flow cytometric analyses of various scIA^{g7} constructions. The scIA^{g7} constructs were grown in SD-CAA (2% dextrose, 0.67% yeast nitrogen base, 1% Casamnino acids (CAA, Difco, Detroit, Mich.)) at 30° C. for 18-20 h. To induce surface scIA^{g7} expression, yeast cells were pelleted by centrifugation, resuspended to an OD₆₀₀ of about 1.0 in SG-CAA(2% galactose, 0.67% yeast nitrogen base, 1% Casamino acids), and incubated at 20° C. After ˜18-20 h of incubation, cultures were harvested, approximately 10⁷ cells/tube were incubated on ice, washed with PBS (10 mM NaPO₄, 150 mM NaCl, pH 7.3) containing 0.5% bovine serum albumin (BSA), and incubated for 1 hr with 25 μL of 10 μg/mL anti-HA mAb 12CA5 (Boehringer Mannheim, Indianapolis, Ind.), anti-c-myc mAb 9E10 (1:50 dilution of raw ascites fluid; Berkeley Antibody Co., Richmond, Calif.), or 10 μg/ml anti-IA^{g7}mAb 10.216 purified from hybridoma supernatant. Cells were washed with PBS and incubated for 1 h on ice with FITC-labeled F(ab′)₂ goat anti-mouse IgG (1:50) (Kirkegaard and Perry Labs, Inc., Gaithersburg, Md.). Labeled cells were washed with PBS and analyzed on a Coulter Epics XL flow cytometer. As observed in the flow histograms, the Aga-2-IA^{g7} fusion in single chain format (Aga-2-HA-β-chain-linker-α-chain-c-myc) was not detectable on the yeast, which is consistent with the instability of the class II product. The Aga-2-IA^{g7} fusion with 3 peptides (GAD65[78-96], B9-23 [Insulin], BDC2.5 [alanine stabilized variant]) linked at the IA^{g7} amino terminus of the β-chain exhibited low or undetectable levels of expression.

L-cells transfected with I-A^{g7} were tested for the direct binding of the biotinylated-1-A^{g7} specific peptide 1040-63 (B-1-4-063, Biotin-RTRPLWVRME (RTRPLWVRME, SEQ ID NO:27). Transfected and non-transfected L-cells were grown until confluent and harvested. L-cells (˜2×10⁵ cells/aliquot) were incubated with varying concentrations of the B-1040-63 peptide for 3 hours at 37° C. in PBS/0.5% BSA, pH 6. Aliquots of cells were washed three times with PBS/0.5% BSA, pH 7, and incubated for one hour with streptavidin-phycoerythrin (PE) conjugate (1:200 dilution; PharMingen, SanDiego, Calif.). L-cells were then washed three times inPBS/0.5% BSA, pH 7, and analyzed by fluorescence activated cell sorting using a Coulter Epics XL flow cytometer as described herein. FIG. 13 is a binding curve showing I-A^{g7}-positive L-cell binding to titrated amounts of B-1040-63 peptide (1.6 μM-50 μM). At the peptide concentrations tested, non-specific binding of B-104-63 to I-A^{g7}-negative L-cells does not occur.

### Example 3

### Mutagenesis of Class II MHC Fusion Proteins

The production of mutagenic libraries in order to generate stabilized MHC Class II, A^{g7} has been described. The random mutagenesis strategy employed the use of error-prone PCR and yeast homologous recombination to generate mutagenic libraries. The scIA^{g7} constructs were amplified using the flanking AGA-2-specific upstream primer I (splice 4/L, 5′-GGC AGC CCC ATA AAC ACA CAG TAT-3′, SEQ ID NO:10) and downstream primer 4 (T7 Promoter, 5′-TAA TAC GAG TCA CTA TAG GG-3′, SEQ ID NO:11) with an additional ˜100 bp upstream and ˜300 bp downstream extending into the display vector (pcT302). Random nucleotide errors were i incorporated into scIA^{g7} constructs using Taq polymerase (Gibco BRL/Invitrogen, Carlsbad, Calif.) in the presence of 2 mM MgCl₂ and 0.3 mM MnCl₂. The directed mutagenesis strategy utilized PCR sewing and yeast homologous recombination to mutate the β56 and β57 residues of scIA^{g7} β-chain. This process involved: a PCR sewing step with primer 3 (5′-TAC CGC GCG GTG ACC GAG CTC GGG CGG NNS NNS GCC GAG TAC TAC AAT AAG C-3′, SEQ ID NO:12) degenerate (where N is any nucleotide and S is C or G) at each position to be varied; reverse primer 2 (5′-CCG CCC GAG CTC GGT CAC CGC GCG GTA CTC GCC CAC GTC G-3′, SEQ ID NO:13) complementary to the 18 bases at the 5′ end of this primer; and primer 1 (splice 41L) and primer 4 (T7 promoter) flanking primers that amplify the entire scIA^{g7} construct. The underlined bases in primer 3 and primer 2 indicate the position of a silent mutation introducing a SacI restriction site into the construct. Approximately 150 ng of linear random or directed mutagenic PCR product and ˜150 ng of Nhe1-BglII-digested 7M sc4F10 pCT302 were combined (per transformation) and transformed into S. cerevisae EBY100 yeast by electroporation to generate libraries, with selection for growth in the absence of tryptophan. Transformants were pooled into ˜250-mil SD-CAA and grown ˜48 hr at 30° C. Isolated clones from each mutagenic strategy were rescued and sequenced to verify mutagenesis. Approximately 4-7 nucleotide errors were incorporated per 1000 base pairs in the random scIA^{g7} libraries.

### Example 4

### Production of Mutant Libraries of scMHC Class II Protein

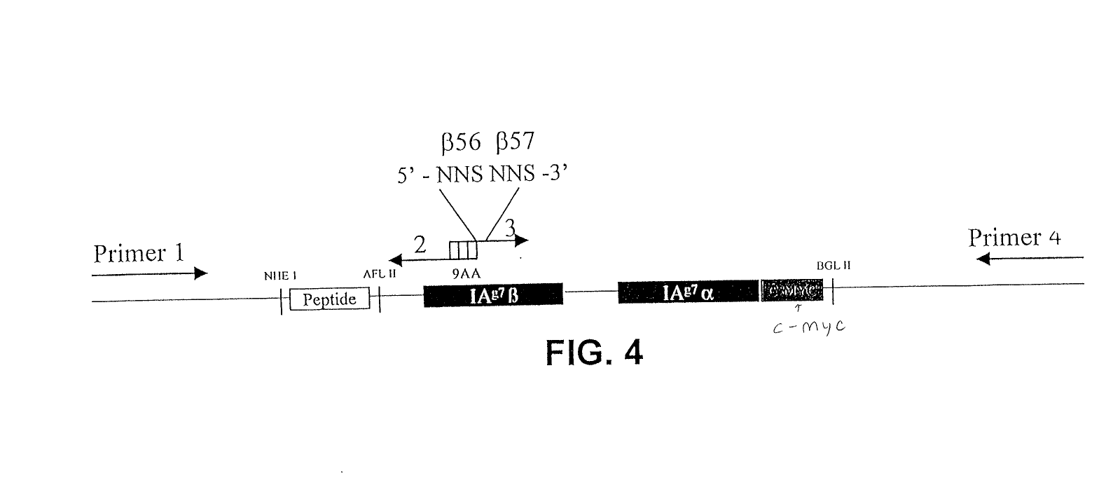

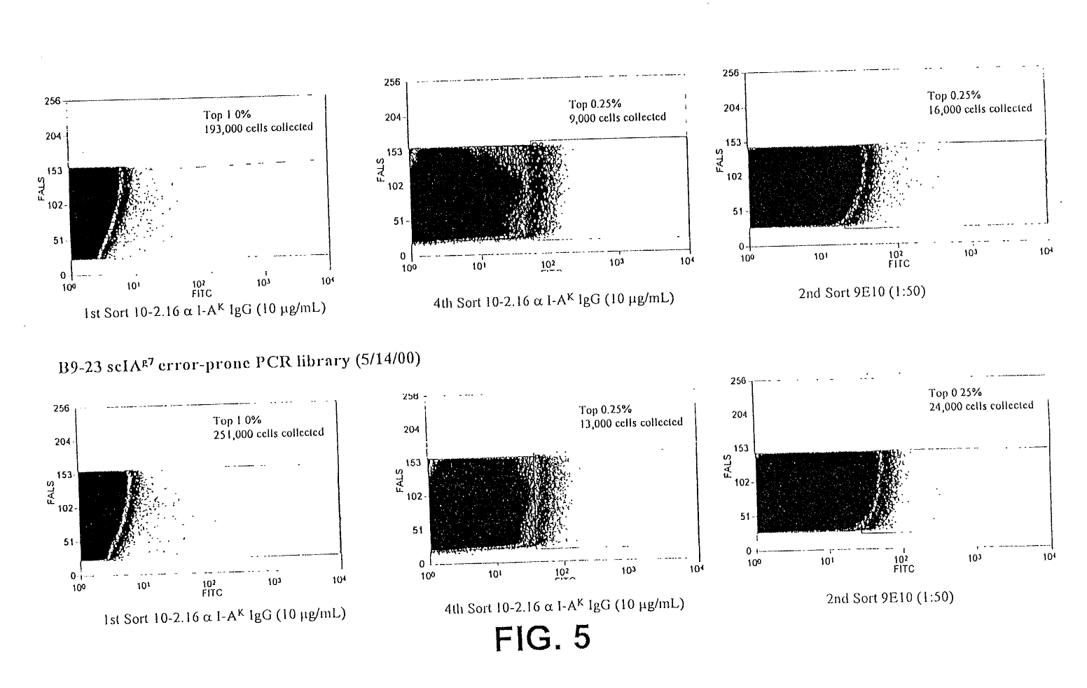

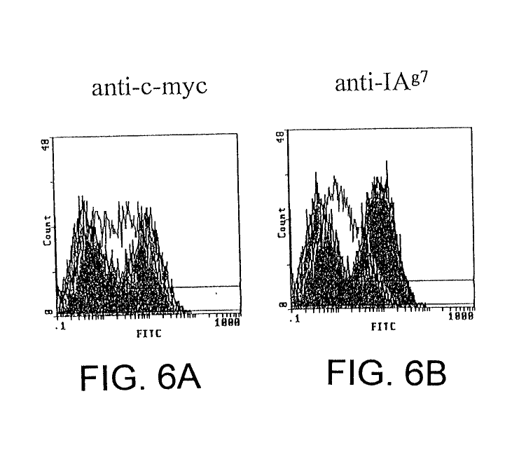

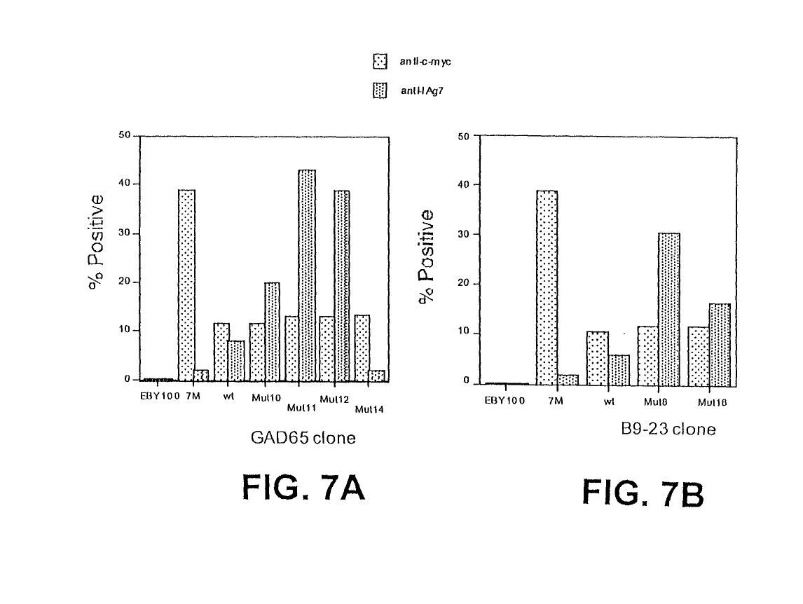

In order to generate stabilized MHC Class II, A^{g7}, the random mutagenesis strategy summarized in FIG. 4 was employed. This strategy used error-prone PCR and yeast homologous recombination to generate mutagenic libraries. The scIA^{g7} constructs were amplified using the flanking AGA2-specific upstream primer 1 (splice 4/L, 5′-GGC AGC CCC ATA AAC ACA CAG TAT-3′, SEQ ID NO:14) and downstream primer 4 (T7 Promoter, 5′-TAA TAC GAC TCA CTA TAG GG-3′, SEQ ID NO:15) with an additional ˜100 bp upstream and ˜300 bp down stream extending into the display vector (pCT302). Random nucleotide errors were incorporated into scIA^{g7} constructs using Taq polymerase (Gibco BRL/Invitrogen, Carlsbad, Calif.) in the presence of 2 mM MgCl₂ and 0.3 mM MnCl₂. The directed mutagenesis strategy utilized PCR sewing and yeast homologous recombination to mutate the β56 and β57 residues of scIA^{g7} β-chain. This process involved: a PCR sewing step with primer 3 (5′-TAC CGC GCG GTG ACC GAG CTC GGG CGG NNS NNS GCC GAG TAC TAC AAT AAG C-3′, SEQ ID NO: 12) degenerate (N is any nucleotide and S is C or G) at each position to be varied; reverse primer 2 (5′-CCG CCC GAG CTC GGT CAC CGC GCG GTA CTC GCC CAC GTC G-3′, SEQ ID NO:13) complementary to the 18 bases at the 5′ end of this primer; and primer 1 (splice 4/L) and primer 4 (T7 promoter) flanking primers that amplify the entire scIA^{g7}β56β57 construct. Underlined bases in primer 3 and primer 2 indicate the position of a silent mutation introducing a SacI restriction site into the construct. Approximately 150 ng random or directed mutagenic PCR product and ˜150 ng of Nhe1-BglII-digested 7M sc4F10 pCT302 were combined (per transformation) and transformed into S. cerevisiae EBY100 yeast by electroporation to generate libraries. Transformants were pooled into ˜250-mil SD-CAA and grown 48 hr at 30° C. Isolated clones from each mutagenic strategy were rescued and sequenced to verify mutagenesis. Approximately 4-7 nucleotide errors were incorporated per 1000 base pairs in the random scIA^{g7} libraries.

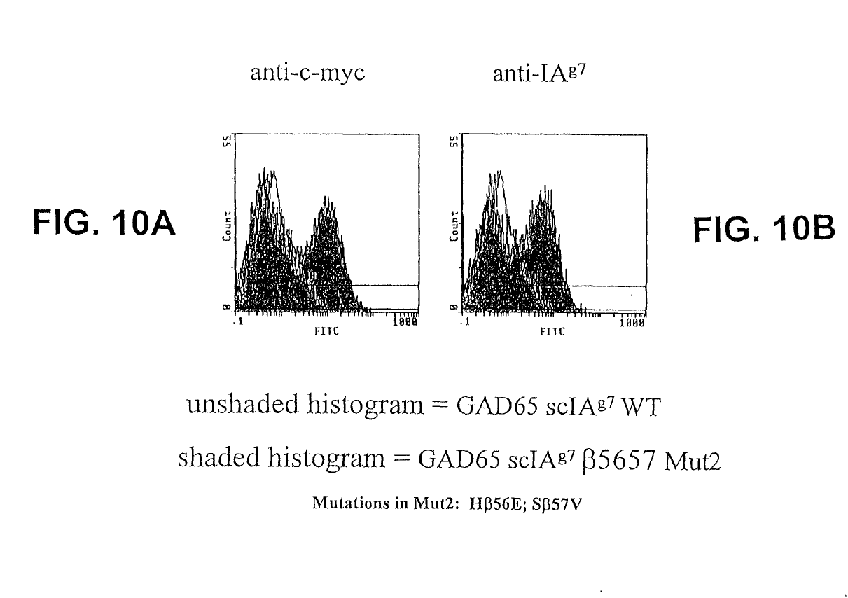

Yeast displaying the GAD65scIA^{g7}WT and GAD65 scIA^{g7}β5657Mut2 fusion proteins were analyzed by flow cytometry. GAD65scIA^{g7}WT/yeast and GAD65 scIA^{g7}β5657Mut2/yeast cells were stained with anti-IA^{g7}mAb 10.216 and anti-c-mycmAb 9E10 followed by FITC-labeled F(ab′)₂ goat anti-mouse IgG. Labeled cells were analyzed on a Coulter Epics XL flow cytometer. The resultant GAD65scIA^{g7}WT (unshaded) and GAD65 scIA^{g7}β5657Mut2 (shaded) histograms are shown in FIGS. 10A-10B. A positive population shift of the GAD65 scIA^{g7}β5657Mut2/yeast was observed when compared to the GAD65scIA^{g7}WT/yeast indicating an increased surface levels (i.e. increased stability). GAD65 scIA^{g7}β5657Mut2 contained Hβ56E and Sβ57V mutations, as determined by sequence analysis.

### Example 5

### Flow Cytometric Analysis of Mutant Libraries

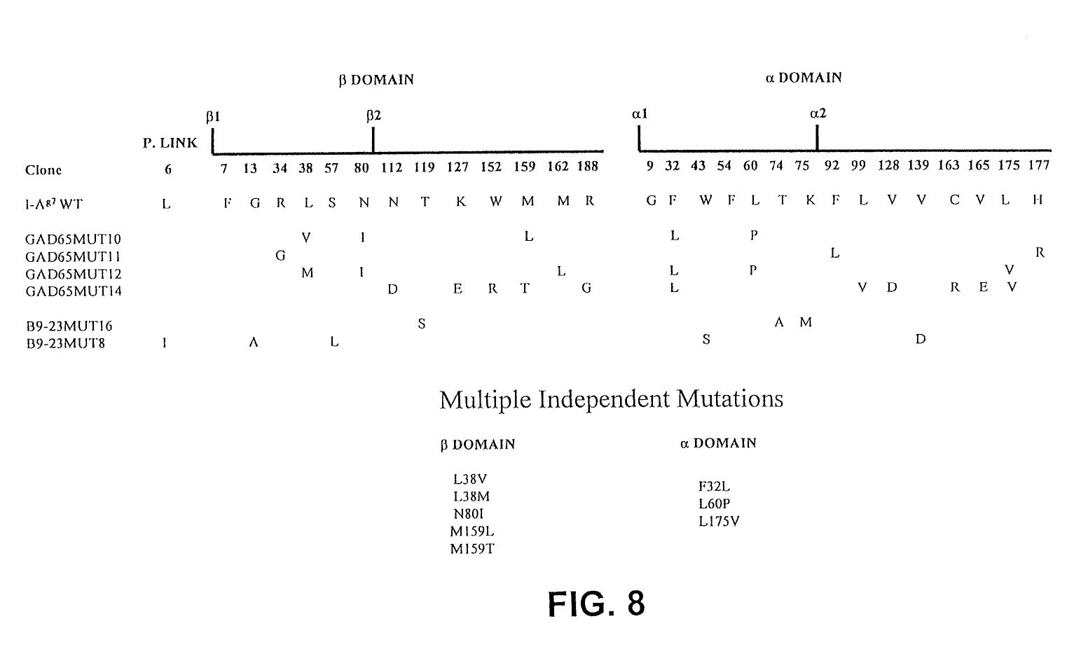

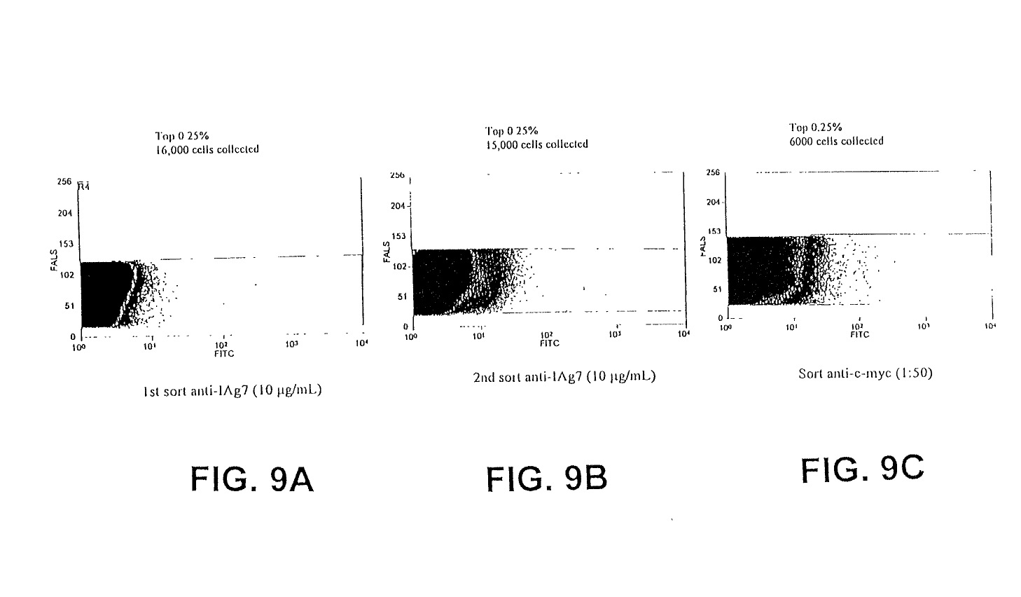

Yeast displaying the B9-23scIA^{g7}wild-type(WT) and B9-23scIA^{g7}Mut8 Aga-2 fusions were analyzed by flow cytometry. B9-23scIA^{g7}WT/yeast and B9-23scIA^{g7}Mut8/yeast were stained with anti-IA^{g7} mAb 10.216 and anti-c-myc mAb 9E10 followed by FITC-labeled F(ab′)₂ goat anti-mouse IgG. Labeled cells were analyzed on a Coulter Epics XL flow cytometer. The resultant B9-23scIA^{g7}WT (unshaded) and B9-23scIA^{g7}Mut8 (shaded) histograms are shown in FIG. 8. A positive population shift of the B9-23scIA^{g7}Mut8/yeast was observed when compared to the B9-23scIA^{g7}WT/yeast indicating increased surface levels (i.e. increased stability). A negative population has been observed for all yeast-displayed proteins. Without wishing to be bound by any particular theory, we believe that this is caused by yeast at a stage of growth or induction incapable of expressing the surface fusion protein. B9-23scIA^{g7}Mut8 was determined to have the following mutations: Gβ13A, Sβ57L, Wα43S, and Vα139D.

### Example 6

### Analysis of Clones Isolated by Sorting

The mutant clones isolated by cell sorting from the GAD65(78-96) scIA^{g7} and the B9-23 scIA^{g7} error-prone PCR libraries were further analyzed. Binding levels are shown as % positive population shift to anti-c-myc mAb and anti-IA^{g7} mAb for GAD65(78-96) scIA^{g7} and B9-23 scIA^{g7} clones isolated. Six mutants in addition to GAD65(78-96) scIA^{g7}WT/yeast, B9-23 scIA^{g7}WT/yeast, 7Msc4F10/yeast (anti-c-myc mAb positive control), and S. cerevisiae EBY100 (negative control) were induced in galactose medium overnight at 30° C. Cells were analyzed by flow cytometry after staining with anti-c-myc followed by FITC-labeled F(ab′)₂ goat anti-mouse IgG (more shaded bars), or stained with anti-IA^{g7} mAb followed by FITC-labeled F(ab′)₂ goat anti-mouse IgG (less shaded bars). Mutants isolated yielded higher surface level from the FACS procedure binding to anti-c-myc and anti-IA^{g7} antibodies than wild-type counterparts (except for GAD65Mut14). This correlates with increased protein stability. GAD65Mut14 has previously shown higher surface display than GAD65WT. Low expression levels in this study could possibly be attributed to experimental error.

### Example 7

### Rapid (One Day) Sequential Sorting of BDC2.5

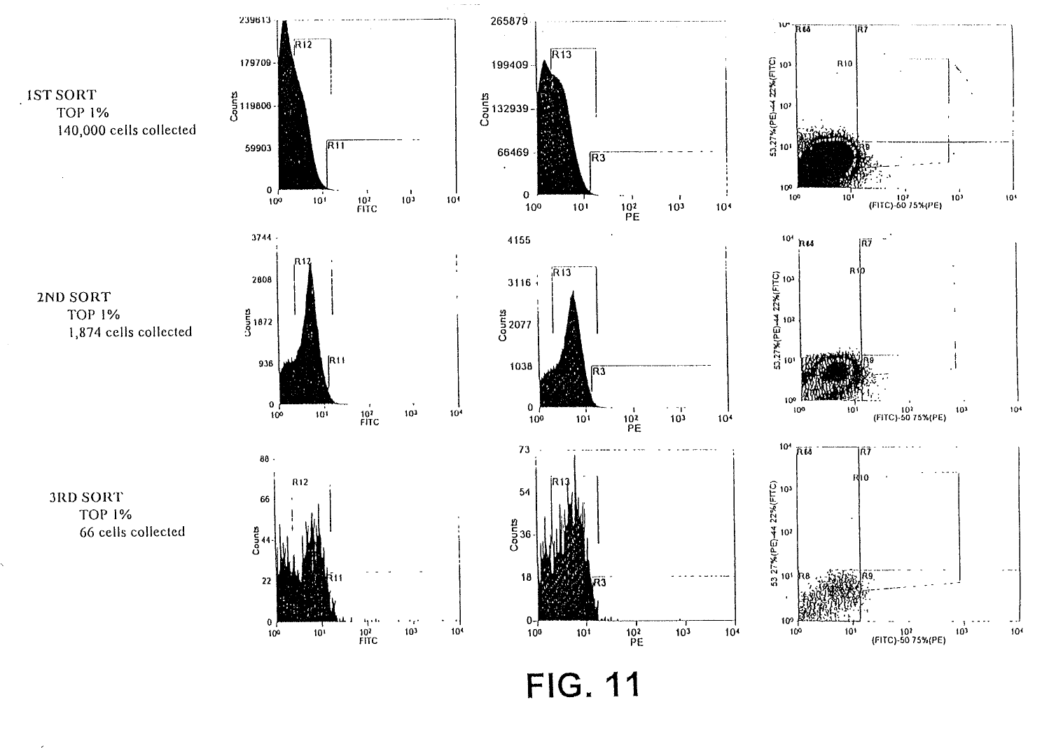

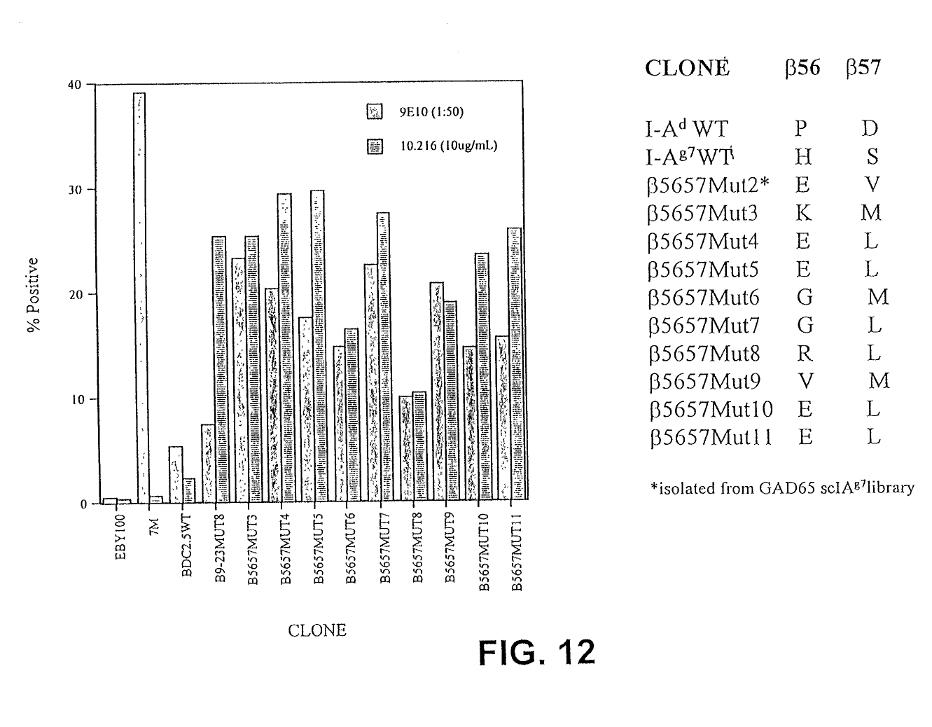

The BDC2.5 scIA^{g7}β5657 yeast library was stained with 12.5 μl anti-lag mAb 10.216 (10 μg/ml) and 12.5 μl biotin-labeled anti-c-myc mAb 9E10 (1:100) (Berkeley Antibody Co., Richmond, Calif.), washed with buffer (PBS/0.5% BSA), and incubated with 12.5μl FITC-labeled F(ab′)₂ goat anti-mouse, γ_{2b} chain specific, IgG_{2b} (1:50) (Southern Biotechnology Associates, Inc., Birmingham, Ala.) and streptavidin-phycoerythrin (SA-PE) conjugate (1:100) (PharMingen, San Diego, Calif.). After washing, samples were sorted in purification mode (coincident negative cells rejected) on a Cytomation MoFlo sorter (Cytomation, Fort Collins, Colo.). A total of 2×10⁷ cells were examined during the first sorting round, collecting ˜1% of the population. The collected cells were sequentially sorted twice more on the same day collecting the top ˜1% of the population. The cells collected from the third sort were plated onto selective glucose plates and grown for ˜48 h. Isolated clones were further examined by flow cytometry. See FIG. 11.

### Example 8

### Summary of Clones Isolated by Sorting from BDC2.5 scIA^{g7}β5657 Library

Binding levels are shown as a % positive population shift to anti-c-myc mAb and anti-IA^{g7} mAb from BDC2.5 scIA^{g7}β5657 clones isolated from the final sequential sort. Nine mutants in addition to BDC2.5 scIA^{g7}WT/yeast, B9-23 scIA^{g7} Mut8/yeast (anti-IA^{g7} mAb positive control), 7Msc4F10/yeast (anti-c-myc mAb positive control), and EBY 100 (negative control) yeast were induced in galactose medium overnight at 30° C. Cells were analyzed by flow cytometry after staining with anti-c-myc mAb (stippled bars), or stained with anti-IAg7 mAb (shaded bars) followed by FITC-labeled F(ab′)₂ goat anti-mouse IgG. Mutants isolated yielded higher surface level binding to anti-c-myc and anti-IA^{g7} antibodies than its BDC2.5 scIA^{g7}WT counterpart. BDC2.5 scIA^{g7}β5657 mutants were sequenced and contained consensus motifs of E/G₅₆ and L/M₅₇.

### Example 9

### Engineering of Single-Chain I-Ag^{g7} β₁α₁ MHC Class II Fusion Proteins

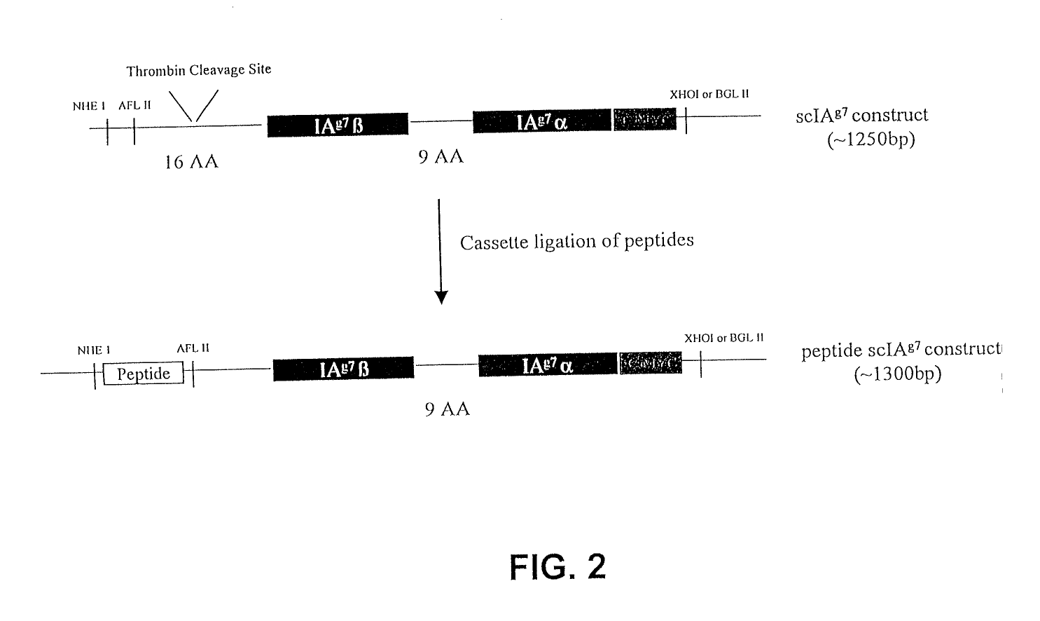

To generate scI-A^{g7} β₁α₁ fusions (Aga-2-β₁-domain-α₁-domain-c-myc), the scI-A^{g7} constructs (FIG. 2) were amplified through a process termed PCR sewing as described herein. Briefly, an Aga-2 specific upstream primer (splice 4/L, SEQ ID NO:14) and a downstream I-Ag^{g7}β₁ domain specific primer (B1A1 reverse-sew, 5′-TAC GTG GTC GGC CTC AAT GTC GTC TTC AAG CCG CCG CAG GGA GGT GGG GAC CTC-3′, SEQ ID NO:25) were used to amplify the 5′ end and β₁ domains of the sc-A^{g7} constructs. An I-A^{g7}α₁ domain specific primer (B1A1 forward-sew, 5′-GAG GTC CCC ACC TCC CTG CGG CGG CTT GAA GAC ATT GAG GCC GAC CAC GTA-3′, SEQ ID NO:27), containing 18 bases of the 5′ end complementary to the previous primer, and a primer coding for the 3′ end of the α₁ domain and c-myc epitope tag (A1-reverse-c-myc-stop, 5′-CAA TAG AGA TCT TTA TCA CAA GTC TTC TTC AGA AAT AAG CTT TTG TTC ATT GGT AGC TGG GGT GAA ATT TGA CCT C-3′, SEQ ID NO:26) were used to amplify the entire α₁ domain. The resultant PCR products were mixed and PCR amplified using Splice 4/L and A1-reverse-c-myc-stop flanking primers to generate the full-length fusion constructs. The scI-A^{g7} β₁α₁ fusion PCR-products were digested with Nhe1 and BglII and ligated into the Nhe1 and BglII-digested yeast surface display vector, pCT302. The ligation mix was transformed into E coli DH10B, and rescued plasmid DNA was then used to transform S. cerevisiae strain EBY100 as described herein. Shown are schematics of several scI-A^{g7} β₁α₁ fusions that were generated, including the various peptide sequences incorporated at 5′ end of the 48 base-pair linker. See FIG. 14.

### REFERENCES

Boder, E. T., and Wittrup, K. D. (1997) Nat. Biotech. 15, 553-557.

Bolin, D. R. et al. (2000) J. Med. Chem. 43(11), 2135-2148.

Bouvier, M., and Wiley, D. C. (1994) Science 265(5170), 398-402.

Brusic, V. et al. (1998) Bioinformatics 14(2), 121-130.

Carrasco-Marin, E. et al. (1996) J. Immunol. 156(2), 450-458.

Castano, A. R. et al. (1995) Science 269(5221), 223-226.

Chang, J. W. et al. (2001) J. Biol. Chem. 276, 24170-24176.

Cho, B. K. et al. (1998) J. Immunol. Methods 220(1-2), 179-188.

Crawford, F. et al. (1998) Immunity 8(6), 675-682.

Cresswell, P. (1994) Annu. Rev. Immunol. 12, 259-293.

Davenport, M. P. et al. (1996) Immunology 88(4), 482-486.

Davis, G. T. et al. (1991) Biotechnology 9(2), 165-169.

Engelhard, V. H. (1994) Curr. Op. Immunol. 6, 13-23.

Falcioni, F. et al. (1999) Nat. Biotechnol. 17(6), 562-567.

Falk, K. et al. (1991) Nature 351(6324), 290-296.

Fremont, D. H. et al. (1996) Science 272(5264), 1001-1004.

Geitz, R. D. et al. (1995) Yeast 11, 355-360.

Goldstein, J. S. et al. (1998) J. Immunol. 160(7), 3180-3187.

Gregori, S. et al. (2000) Int. Immunol. 12(4), 493-503.

Hammer, J. et al. (1992) J. Exp. Med. 176(4), 1007-1013.

Hammer, J. et al. (1993) Cell 74(1), 197-203.

Harrison, L. C. et al. (1997) J. Exp. Med. 185(6), 1013-1021.

Howard, M. C. et al. (1999) Immunol. Today 20(4), 161-165.

Ignatowicz, L. et al. (1995) J. Immunol. 154(8), 3852-3862.

Ishioka, G. Y. et al. (1994) J. Immunol. 152(9), 4310-4319.

Judkowski, V. et al. (2001) J. Immunol. 166, 908-917.

Kozono, H. et al. (1994) Nature 369, 151-154.

Kranz, D. M. et al. (1984) Proc. Natl. Acad. Sci. USA 81, 7922-7926.

Lamont, A. G. et al. (1990) J. Immunol. 144, 2493.

Le Doussal, J. et al. (2000) J. Immunol. Methods 241(1-2), 147-58.

Lepley, D. M. et al. (1997) Transplantation 63(5), 765-774.

Madden, D. R. (1995) Annu. Rev. Immunol. 13, 587-622.

Mescher, M. F. et al. (1983) Methods Enzymol. 92, 86-109.

Milik, M. et al. (1998) Nat. Biotechnol. 16(8), 753-756.

Mottez, E. et al. (1995) J. Exp. Med. 181(2), 493-502.

Onda, T. et al. Mol. Immunol. 32(17-18), 1387-1397.

Rammensee, H. G. et al. (1995) Immunogenetics 41(4), 178-228.

Raymond, C. K. et al. (1999) Biotechniques 26(1), 134-138, 140-141.

Reay, P. A. et al. (1992) EABO J. 11(8), 2829-2839.

Ribaudo, R. K., and Margulies, D. H. (1995) J. Immunol. 155, 3481-3493.

Ridgway, W. M. et al. (1999) Science 284(5415), 749-751.

Rothbard, J. B. (1991) Annu. Rev. Immunol. 9, 527-565.

Rudensky, A. et al. (1992) Nature 359(6394), 429-431.

Sakita, I. Et al. (1996) J. Immunol. Methods 192(1-2), 105-115.

Schmitt, L. et al. (1999) J. Mol. Biol. 286(1), 207-218.

Scott, C. A. et al. (1998) [published erratum appears in Immunity 1998 Apr;8(4):531]. Immunity 8(3), 319-329.

Sette, A. et al. (1994) Annu. Rev. Immunol. 12, 413-431.

Shields, M. J. et al. (1998) J. Immunol. 160(5), 2297-2307.

Shields, M. J. et al. (1999) Mol. Immunol. 36(9), 561-573.

Shirai, M. et al. (1997) J. Immunol. 158(7), 3181-3188.

Shusta, E. V. et al. (1999) J. Mol. Biol., 292, 949-956.

Smith, J. D. et al. (1995) Mol. Immunol. 32(7), 531-540.

Stern, L. J. et al. (1994) Nature 368, 215-221.

Stern, L. J., and Wiley, D. C. (1992) Cell 68(3), 465-477.

Udaka, K. et al. (1996) J. Immunol. 157, 670-678.

Uger, R. A. et al. (1999) J. Immunol. 162(10), 6024-6028.

Van der Burg, S. H. et al. (1996) J. Immunol. 156, 3308-3314.

Wucherpfennig, K. W. et al. (1995) Proc. Natl. Acad. Sci. USA 92(25), 11935-11939.

Zhu, X. et al. (1997) Eur. J. Immunol. 27(8), 1933-1941.

TABLE 1
Results of Mutagenic Libraries

| | # Transformants |
|---|---|
| Random Mutagenesis | |
| scIA^{g7} | 1.7 × 10⁶ |
| BDC2.5(A) scIA^{g7} | 1.4 × 10⁶ |
| B9-23 scIA^{g7} | 1.2 × 10⁶ |
| GAD65(78-96 scIA^{g7} | 1.2 × 10⁶ |
| BDC2.5(A) scIA^{g7} #1 | 3.0 × 10⁵ |
| BDC2.5(A) scIA^{g7} #2 | 3.7 × 10⁶ |
| Directed Mutagenesis (β56/57) | |
| GAD65(78-96 scIA^{g7} β5657 | 4.3 × 10⁴ |
| BDC2.5(A) scIA^{g7} β5657 | 1.0 × 10⁵ |

TABLE 2
Nucleotide and Amino Acid Sequences
Nucleotide and amino acid sequences for the scI-A^{g7} construct
(α-chain-linker-β-chain-c-myc) and 3 peptides (GAD65[78-96],
B9-23[Insulin], BDC2.5 [alanine stabilized variant]).
scI-A^{g7} construct (α-chain-linker-α-chain-c-myc)

nucleotide sequence (SEQ ID NO: 16)
GCTAGCGGTGGACTTAAGGGTGGCGGCGGTTCTTTAGTTCCAAGAGGTTCTGGTGGCGGTGG
CTCTGGAGACTCCGAAAGGCATTTCGTGCACCAGTTCAAGGGCGAGTGCTACTTCACCAACG
GGACGCAGCGCAThCGGCTCGTGACCAGATACATCTACAACCGGGAGGAGTACCTGCGCTTC
GACAGCGACGTGGGCGAGTACCGCGCGGTGACCGAGCTGGGGCGGCACTCAGCCGAGTACTA
CAATAAGCAGTACCTGGAGCGAACGCGGGCCGAGCTGGACACGGCGTGCAGACACAACTACG
AGGAGACGGAGGTCCCCACCTCCCTGCGGCGGCTTGAACAGCCCAATGTCGCCATCTCCCTG
TCCAGGACAGAGGCCCTCAACCACCACAACACTCTGGTCTGTTCGGTGACAGATTTCTACCC
AGCCAAGATCAAAGTGCGCTGGTTCAGGAATGGCCAGGAGGAGAAGTGGGGGTCTCATCCAC
ACAGCTTATTAGGAATGGGGACTGGACCTTCCAGGTCCTGGTCATGCTGGAGATGACCCCTC
ATCAGGGAGAGGTCTACACCTGCCATGTGGAGCATCCCAGCCTGAAGAGCCCCATCACTGTG
GAGTGGAGGGGCGGAGGAGGTTCTGGAGGTGGCGGAGAAGACGACATTGAGGCCGACCACGT
AGGCTTCTATGTACAACTGTTTATCAGTCTCCTGGAGACATTGGCCAGTACACACATGAATT
TGATGGTGATGAGTTGTTCTATGTGGACTTGGATAAGAAGAAAACTGTCTGGAGGCTTCCTG
AGTTTGGCCAATTGATACTCTTTGAGCCCCAAGGTGGACTGCAAAACATAGCTGCAGAAAAA
CACAACTTGGGAATCTTGACTAAGAGGTCAAATTTCACCCCAGCTACCAATGAGGCTCCTCA
AGCGACTGTGTTCCCCAAGTCCCCTGTGCTGCTGGGTCAGCCCAACACCCTTATCTGCTTTG
TGGACAACATCTTCCCACCTGTGATCAACATCACATGGCTCAGAAATAGCAAGTCAGTCACA
GACGGCGTTTATGAGACCAGCTTCCTCGTCAACCGTGACCATTCCTTCCACAAGCTGTCTTA
TCTCACCTTCATCCCTTCTGATGATGACATTTATGACTGCAAGGTGGAGCACTGGGGCCTGG
AGGAGCCGGTTCTGAAACACTGGGAACAAAAGCTTATTTCTGAAGAAGACTTGTGATAAAGATCT
amino acid sequence (SEQ ID NO: 17)
ASGGLKGGGGSLVPRGSGGGGSGDSERHFVHQFKGECYFTNGTQRIRLVTRYIYNREEYLRF
DSDVGEYRAVTELGRHSAEYYNKQYLERTRAELDTACRHNYEETEVPTSLRRLEQPNVAISL
SRTEALNHHNTLVCSVTDFYPAKIKVRWFRNGQEETVGVSSTQLIRNGDWTFQVLVMLEMTP
HQGEVYTCHVEHPSLKSPITVEWRGGGGSGGGGEDDIEADHVGFYGTTVYQSPGDIGQYTHE
FDGDELFYVDLDKKKTVWRLPEFGQLILFEPQGGLQNIAAEKHNLGILTKRSNFTPATNEAP
QATVFPKSPVLLGQPNTLICFVDNIFPPVINITWLRNSKSVTDGVYETSFLVNRDHSFHKLS
YLTFIPSDDDIYDCKVEHWGLEEPVLKHWEQKLISEEDL

BDC2.5(A) Peptide
CODING sequence (SEQ ID NO: 18)
GGTAAAAAGGTTGCTGCACCAGCTTGGGCTCGTATGGGT
amino acid sequence (SEQ ID NO: 19)
GKKVAAPAWARMG
GAD65(78-96) Peptide
coding sequence (SEQ ID NO: 20)
AAACCATGTAATTGTCCAAAAGGTGATGTTAATTATGCTTTTTTGCATGCTACTGAT
amino acid sequence (SEQ ID NO:22)
KPCNCPKGDVNYAFLHATD
B9-23 Insulin Peptide
coding sequence (SEQ ID NO: 23)
TCTCATTTGGTTGAAGCTTTGTATTTGGTTTGTGGTGAAAGAGGT
amino acid sequence (SEQ ID NO: 24)
SHLVEALYLVCGERG

What is claimed is:

1. A mutagenized combinatorial library of Major Histocompatibility Complex (MHC) Class II chimeric proteins displayed on the surfaces of recombinant yeast cells, wherein the mutagenized combinatorial library comprises at least one member MHC Class II protein which is improved in conformational stability or in peptide binding or T cell receptor binding as compared with a comparison MHC Class II protein which has not been mutagenized.

2. The mutagenized combinatorial library of claim 1 wherein the MHC Class II chimeric protein is a chimeric protein, said chimeric protein comprising a portion mediating binding to the surfaces of the recombinant yeast cells and a portion which comprises a peptide binding region of a MHC Class II protein.

3. The mutagenized combinatorial library of claim 2 wherein the portion mediating binding to the surfaces of the recombinant yeast cells is a mating adhesion receptor portion.

4. The mutagenized combinatorial library of claim 3 wherein the mating adhesion receptor portion is an AGA2 portion.

5. The mutagenized combinatorial library of any of claims 2 to 4 wherein the chimeric protein further comprises a portion comprising an amino acid sequence of a peptide which binds to the peptide binding region of the MHC Class II protein.

6. The mutagenized combinatorial library of any of claims 2 to 5 wherein the chimeric protein further comprises a portion derived from a c-myc protein and which mediates binding to a c-myc specific antibody.

7. The mutagenized combinatorial library of any of claims 2 to 6 wherein a peptide which binds to the peptide binding region of the MHC Class II chimeric protein is associated with an autoimmune disease.

8. The mutagenized combinatorial library of claim 6 wherein the autoimmune disease is insulin dependent diabetes mellitus.

9. The mutagenized combinatorial library of claim 8 wherein the peptide binding region specifically binds a peptide having the amino acid sequence given in SEQ ID NO:19, SEQ ID NO:22 or SEQ ID NO:24.

10. The mutagenized combinatorial library of claim 6 wherein said chimeric protein comprises an amino acid sequence as given in SEQ ID NO:17.

11. An isolated mutant MHC Class II chimeric protein, wherein said protein comprises a portion mediating binding to the surfaces of the recombinant yeast cells and a portion which comprises a peptide binding region of a MHC Class II protein and wherein said chimeric protein is improved in stability or in T cell receptor binding as compared with an MHC Class II chimeric protein which is not a mutant chimeric protein.

12. The isolated mutant MHC Class II chimeric protein of claim 11 wherein the chimeric protein further comprises a portion comprising an amino acid sequence of a peptide which binds to the peptide binding region of the MHC Class II protein.

13. The isolated mutant MHC Class II chimeric protein of claim 11 wherein the peptide which binds to the peptide binding region of the MHC Class II protein is associated with an autoimmune disease.

14. The isolated mutant MHC Class II chimeric protein of claim 13 wherein the autoimmune disease is insulin dependent diabetes mellitus, and wherein the portion mediating binding to the surfaces of the recombinant yeast cells is a mating adhesion receptor portion.

15. The isolated mutant MHC Class II chimeric protein of claim 11 wherein the peptide binding region specifically binds a peptide having the amino acid sequence given in SEQ ID NO:19, SEQ ID NO:22 or SEQ ID NO:24.

16. The isolated mutant MHC Class II chimeric protein of claim 12 wherein said chimeric protein further comprises a detectable label.

17. The isolated mutant MHC Class II chimeric protein of claim 16 wherein the detectable label is a fluorescent moiety, a chromophore, a radionuclide, a chemiluminescent agent, a magnetic particle, an enzyme, a cofactor, a substrate or a toxin.

18. A method for detection of a lymphocyte having a T cell receptor protein in a biological sample, said method comprising the steps of contacting the sample with an isolated mutant chimeric protein of claim 16, wherein said chimeric protein is complexed to the peptide or wherein the chimeric protein and peptide are covalently bound, wherein said chimeric protein comprises a binding region which specifically binds said T cell receptor protein under conditions which allow the binding of the T cell receptor protein to the chimeric protein, and detecting the chimeric protein bound to the T cell receptor protein.

19. The method of claim 18 wherein the biological sample is cells, a tissue sample, biopsy material or bodily fluids.

20. The method of claim 19 wherein detection of the T cell receptor protein is diagnostic of an autoimmune disease.

21. The method of claim 20 wherein the autoimmune disease is selected from the group consisting of insulin dependent diabetes mellitus, multiple sclerosis, Crohn's disease, celiac disease, rheumatoid arthritis and inflammatory bowel disease.

22. A method for treating or preventing an autoimmune disease in person or animal suffering from or susceptible to said autoimmune disease comprising the step of administering to the patient a therapeutically effective amount of an isolated mutant MHC Class II chimeric protein which is improved in conformational stability as compared with a comparison MHC Class II chimeric protein which is not mutant.

23. The method of claim 22 wherein said autoimmune disease is insulin dependent diabetes mellitus.

24. The method of claim 23 wherein said isolated mutant protein has a portion comprising an amino acid sequence as given in SEQ ID NO:17.

25. The method of claim 24 wherein said mutant protein binds a peptide comprising an amino acid sequence as given in SEQ ID NO:19, SEQ ID NO:22 or SEQ ID NO:24.
